(19) United States
(12) Patent Application Publication
(10) Pub. No.: US 2014/0356920 A1
(43) Pub. Date: Dec. 4, 2014

(54) RECOVERY OF HIGHER ALCOHOLS FROM DILUTE AQUEOUS SOLUTIONS

(71) Applicant: Gevo, Inc., Englewood, CO (US)

(72) Inventors: William A. Evanko, Golden, CO (US); Mark Brothers, Longmont, CO (US); Ken Drobish, Castle Rock, CO (US); Aristos A. Aristidou, Highlands Ranch, CO (US); Kent Evans, Littleton, CO (US); Andrew C. Hawkins, Parker, CO (US); Scott Lucas, Shawnee, KS (US)

(73) Assignee: Gevo, Inc., Englewood, CO (US)

(21) Appl. No.: 14/463,429

(22) Filed: Aug. 19, 2014

Related U.S. Application Data
(63) Continuation of application No. 13/586,599, filed on Aug. 15, 2012, now abandoned.
Application No. 13/586,599 is a continuation of application No. 12/824,050, filed on Jun. 25, 2010, now abandoned.
(60) Provisional application No. 61/220,967, filed on Jun. 26, 2009.

(51) Int. Cl. C12P 7/16; B01D 3/06; C07C 29/84

(52) U.S. Cl. CPC C12P 7/16; C07C 29/84; B01D 3/06
USPC 435/160; 435/155; 202/185.1

(57) ABSTRACT

This invention is directed to methods for recovery of C3-C6 alcohols from dilute aqueous solutions, such as fermentation broths. Such methods provide improved volumetric productivity for the fermentation and allow recovery of the alcohol. Such methods also allow for reduced energy use in the production and drying of spent fermentation broth due to increased effective concentration of the alcohol product by the simultaneous fermentation and recovery process which increases the quantity of alcohol produced and recovered per quantity of fermentation broth dried. Thus, the invention allows for production and recovery of C3-C6 alcohols at low capital and reduced operating costs.

### CROSS REFERENCE TO RELATED APPLICATIONS

This application claims the benefit of priority under 35 U.S.C. §119(e) from U.S. Provisional Application No. 61/220,967, filed Jun. 26, 2009, the contents of which are incorporated herein by reference in their entirety.

### FIELD OF THE INVENTION

This application relates generally to methods for recovery of C3-C6 alcohols from dilute aqueous solutions, such as fermentation broths.

### BACKGROUND OF THE INVENTION

Biofuels have a long history ranging back to the beginning of the 20th century. As early as 1900, Rudolf Diesel demonstrated at the World Exhibition in Paris, France, an engine running on peanut oil. Soon thereafter, Henry Ford demonstrated his Model T running on ethanol derived from corn. Petroleum-derived fuels displaced biofuels in the 1930s and 1940s due to increased supply, and efficiency at a lower cost.

Market fluctuations in the 1970s, due the Arab oil embargo and the Iranian revolution, coupled to the decrease in US oil production, led to an increase in crude oil prices and a renewed interest in biofuels. Today, many interest groups, including policy makers, industry planners, aware citizens, and the financial community, are interested in substituting petroleum-derived fuels with biomass-derived biofuels. The leading motivation for developing biofuels is of economical nature, namely, the threat of ‘peak oil’, the point at which the consumption rate of crude oil exceeds the supply rate, thus leading to significantly increased fuel cost results in an increased demand for alternative fuels.

Biofuels tend to be produced with local agricultural resources in many, relatively small facilities, and are seen as a stable and secure supply of fuels independent of geopolitical problems associated with petroleum. At the same time, biofuels enhance the agricultural sector of national economies. In addition, since fossil sources of fuels take hundreds of millions of years to be regenerated and their use increases carbon dioxide levels in the atmosphere, leading to climate change concerns, sustainability is an important social and ethical driving force which is starting to result in government regulations and policies such as caps on carbon dioxide emissions from automobiles, taxes on carbon dioxide emissions, and tax incentives for the use of biofuels.

The acceptance of biofuels depends primarily on economical competitiveness of biofuels when compared to petroleum-derived fuels. Biofuels that cannot compete in cost with petroleum-derived fuels will be limited to specialty applications and niche markets. Today, the use of biofuels is limited to ethanol and biodiesel. Currently, ethanol is made by fermentation from corn in the US, sugar cane in Brazil, and other grains worldwide. Ethanol is competitive with petroleum-derived gasoline, exclusive of subsidies or tax benefits, if crude oil stays above $50 per barrel. Biodiesel has a breakeven price of crude oil of over $60/barrel to be competitive with petroleum-based diesel (Nexant Chain Systems, 2006, Final Report, Liquid Biofuels: Substituting for Petroleum, White Plains, N.Y.).

Several factors influence the core operating costs of a carbohydrate based biofuel source. In addition to the cost of the carbon-containing, plant produced raw material, a key factor in product economic costs for ethanol or other potential alcohol based biofuels, such as butanol, is the recovery and purification of biofuels from aqueous streams. Many technical approaches have been developed for the economic removal of alcohols from aqueous based fermentation media. The most widely used recovery techniques today use distillation and molecular sieve drying to produce ethanol. For example, butanol production via the Clostridia-based acetone-butanol-ethanol fermentation also relied on distillation for recovery and purification of the products. Distillation from aqueous solutions is energy intensive. For ethanol, additional processing equipment to break the ethanol/water azeotrope is required. This equipment, molecular sieves, also uses significant quantities of energy.

Many unit operations have been studied for the recovery and purification of fermentation produced alcohols, including filtration, liquid/liquid extraction, membrane separations (e.g., tangential flow filtration, pervaporation, and perstraction), gas stripping, and “salting out” of solution, adsorption, and absorption. Each of the approaches has advantages and disadvantages depending on the circumstances of the product to be recovered and the product's physical and chemical properties and the matrix in which it resides.

Variables which control the production costs of biofuels can be characterized as those impacting operating costs, capital costs, or both. Typically, key variables that control fermentation economic performance include carbohydrate yield to desired product, product concentration and volumetric productivity. All three key variables, yield, product concentration, and volumetric productivity, impact both capital and operating costs.

As product yield on carbohydrate fermented is increased, the production costs for a given unit of product decrease linearly relative to raw material costs. The product yield on carbohydrate also impacts equipment size, capital expenditures, utilities consumption and feed stock preparation materials such as enzymes, minerals, nutrients (vitamins), and water. For example an increase in product yield on glucose to butanol from 50% to 90% of theoretical results in a 44% decrease in direct operating costs. Also, the increased yield of 90% reduces the amount of raw materials handled and processed. The increased yield directly reduces capital investment required for the production facility as all equipment from carbohydrate preparation through purification and recovery are reduced in size. Equipment, piping, and utility requirements can be reduced by 32% if yield is increased from 50% to 90%. The direct influence of product yield on production costs makes it a key influence on the cost and market viability for biofuels. An approach to increase product yield involves Genetically Engineered Microorganisms (GEMS) that can be constructed to manipulate the organism's metabolic pathway to reduce or eliminate undesired products, increase the efficiency of the desired metabolite or both. This allows for the deletion of one or both of low cost products and undesired products, which increases production of desired products.

For example, US Patent Application Publication 20050089979 discloses a fermentation process that utilizes a Clostridium beijerinckii microorganism that produces a mixture of products including 5.3 g/L acetone, 11.8 g/L butanol, and 0.5 g/L ethanol. An appropriately modified Genetically Engineered Microorganism eliminates acetone and ethanol production while increasing conversion of carbohydrates to butanol. The redirection of a carbohydrate feedstock away from ethanol and acetone to butanol increases butanol production from 11.8 g/L to 18.9 g/L, a 60% increase in butanol production relative to carbohydrate consumption. The elimination of the ethanol and acetone byproducts also allows for reduced capital costs as less equipment is necessary to complete recovery and purification.

Application of biochemical tools, including, genetic engineering and classical strain development can also impact the final product concentration (g/L) and fermentation volumetric productivity (g/L-hr) of the biocatalyst. Final product concentration and volumetric productivity impacts several aspects of product economics, including equipment size, raw material use, and utility costs. As the tolerable product concentration increases in the fermentation, recovery volumes of aqueous solutions are decreased which results in reduced capital costs and smaller volumes of materials to process within the production facility.

Volumetric productivity directly impacts the required fermentor capacity to achieve the same product output. For example, a traditional Clostridium beijerinckii acetone-butanol-ethanol (ABE) fermentation produces a ratio of acetone, butanol, and ethanol. Genetically engineered microbes allow the designed production of a single product, such as n-butanol, isobutanol or 2-butanol (Donaldson et al., U.S. patent application Ser. No. 11/586,315). Butanol tolerant hosts can be identified utilizing techniques to identify and enhance the butanol tolerance (-Bramucci et al., U.S. patent application Ser. No. 11/743,220). These two techniques can then be combined to produce butanol at commercially relative concentrations, and volumetric productivity.

The utilization of GEMs to increase product volumetric productivity and concentration may strongly influence product economics. For example, a butanol fermentation completed at twice the volumetric productivity will reduce fermentor cost by almost 50% for a large industrial biofuels fermentation facility. The fermentor capital cost and size reduction decreases depreciation and operating costs for the facility. Similarly, if the GEMs result in an organism that is tolerant to higher butanol concentrations, operating and capital costs are reduced for a given production volume. For example, if a wild type strain is capable of tolerating 20 g/L butanol and a corresponding genetically improved or genetically enhanced microorganism tolerates 40 g/L butanol, the water load in the fermenter broth volume handled in downstream recovery and purification equipment is reduced by half. In this example, the doubling of product concentration in the fermentation broth almost halves the amount of water to be recovered and processed in recovery unit operations.

A large number of minor cost components also impact operating and capital costs for biofuels production. Example factors that can impact fermentation include, but are not limited to, chemical additives, pH control, surfactants, and contamination are some of the factors but many additional factors can impact fermentation product cost.

### SUMMARY OF THE INVENTION

The present invention describes methods for recovery of C3-C6 alcohols from dilute aqueous solutions, such as fermentation broths, related systems, and methods.

In one embodiment, the invention provides A method to recover a C3-C6 alcohol from a fermentation medium comprising microorganisms, gases and the C3-C6 alcohol, comprising removing at least a portion of the gases from the fermentation medium; increasing the activity of the C3-C6 alcohol in a portion of the fermentation medium to at least that of saturation of the C3-C6 alcohol in the portion, or decreasing the activity of water in a portion of the fermentation medium to at least that of saturation of the C3-C6 alcohol in the portion; forming a C3-C6 alcohol-rich liquid phase and a water-rich liquid phase from the portion of the fermentation medium; and separating the C3-C6 alcohol-rich phase from the water-rich phase.

The method can further comprise culturing a microorganism in the fermentation medium to produce the C3-C6 alcohol and gases; and conducting at least a portion of the water rich phase to the fermentation medium.

The method can further comprise hydrolyzing a feedstock comprising a polysaccharide and at least one other compound to produce fermentable hydrolysis products; fermenting at least a portion of the fermentable hydrolysis products in the fermentation medium to produce the C3-C6 alcohol and gases, wherein the fermentation medium further comprises at least one non-fermented compound; and separating the at least one non-fermented compound from the fermentation medium, or the water-rich phase, or both.

In another embodiment, the invention provides a method to produce a product from a C3-C6 alcohol in a fermentation medium comprising microorganisms, gases and the C3-C6 alcohol, comprising removing at least a portion of the gases from the fermentation medium; distilling a vapor phase comprising water and C3-C6 alcohol from the fermentation medium; reacting the C3-C6 alcohol in the vapor phase to form the product.

The method of claim 1, further comprising culturing a microorganism in a fermentation medium to produce the C3-C6 alcohol and gases; and conducting at least a portion of the water rich liquid phase to the fermentation medium; wherein the step of increasing the activity of the C3-C6 alcohol or decreasing the activity of water further comprises distilling the portion of the fermentation medium to produce a vapor phase comprising water and C3-C6 alcohol and a liquid phase.

In another embodiment, the invention provides a method to recover a C3-C6 alcohol from a dilute aqueous solution that comprises a first amount of the C3-C6 alcohol and gases, comprising removing at least a portion of the gases from the dilute aqueous solution; distilling a portion of the dilute aqueous solution to a vapor phase comprising C3-C6 alcohol and water, wherein the vapor phase comprises between about 1% by weight and about 45% by weight of the first amount of C3-C6 alcohol from the portion of the dilute aqueous solution; and condensing the vapor phase.

In another embodiment, the invention provides a method to operate a retrofit ethanol production plant comprising a pretreatment unit, multiple fermentation units, and a beer still to produce a C3-C6 alcohol, comprising pretreating a feedstock to form fermentable sugars in the pretreatment unit; culturing a microorganism in a fermentation medium comprising the fermentable sugars in a first fermentation unit to produce the C3-C6 alcohol; removing at least a portion of the gases from the fermentation medium; treating a portion of the fermentation medium comprising the C3-C6 alcohol to remove a portion of the C3-C6 alcohol; returning the treated portion of the fermentation medium to the first fermentation unit; and transferring the fermentation medium from the first fermentation unit to the beer still.

In some embodiments, one of the gases is gas is carbon dioxide and in various embodiments, at least about 30% of the carbon dioxide is removed during the step of removing at least a portion of gas from a dilute aqueous solution or fermentation broth, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

The method can further include in the step of removing a step selected from the group consisting of heating, reducing pressure to below atmospheric pressure, adsorption and combinations thereof.

The method can further include in the step of removing reducing pressure to a pressure of between about 1 psia and about 10 psia, or reducing pressure to a pressure of between about 2 psia to about 5 psia.

The method can further include conducting the removed carbon dioxide to a fermentation unit for pH control, venting it or mixtures thereof.

The method can further include treating the gases to remove the C3-C6 alcohol and venting the gases.

The method can further include removing at least one impurity from the fermentation medium or the dilute aqueous solution. The impurity can include ethanol, acetic acid, propanol, phenyl ethyl alcohol or isopentanol.

In another embodiment, the invention provides a method for increasing the concentration of a C3-C6 alcohol in an aqueous solution comprising introducing a first stream of aqueous solution comprising the C3-C6 alcohol into a vessel; subjecting the first stream of aqueous solution comprising the C3-C6 alcohol to reduced pressure to form a vapor comprising the C3-C6 alcohol; contacting the vapor comprising the C3-C6 alcohol with a solution comprising the C3-C6 alcohol to form a condensate comprising condensed vapor of the C3-C6 alcohol, wherein the concentration of the C3-C6 alcohol in the condensate is greater than the concentration of the C3-C6 alcohol in the first stream of aqueous solution.

In another embodiment, the invention provides a method to recover a C3-C6 alcohol from a fermentation medium comprising microorganisms and the C3-C6 alcohol, comprising increasing the activity of the C3-C6 alcohol in a portion of the fermentation medium to at least that of saturation of the C3-C6 alcohol in the portion to form a vapor comprising the C3-C6 alcohol, or decreasing the activity of water in a portion of the fermentation medium to at least that of saturation of the C3-C6 alcohol in the portion to form a vapor comprising the C3-C6 alcohol; condensing the C3-C6 alcohol vapor by contacting the vapor comprising the C3-C6 alcohol with a solution comprising the C3-C6 alcohol; forming a C3-C6 alcohol-rich liquid phase and a water-rich liquid phase from the condensed vapor; and separating the C3-C6 alcohol-rich phase from the water-rich phase.

The method can further comprise culturing a microorganism in the fermentation medium to produce the C3-C6 alcohol; and conducting at least a portion of the water rich phase to the fermentation medium.

The method can further comprise hydrolyzing a feedstock comprising a polysaccharide and at least one other compound to produce fermentable hydrolysis products; fermenting at least a portion of the fermentable hydrolysis products in the fermentation medium to produce the C3-C6 alcohol, wherein the fermentation medium further comprises at least one non-fermented compound; and separating the at least one non-fermented compound from the fermentation medium, or the water-rich phase, or both. method to produce a C3-C6 alcohol, comprising culturing a microorganism in a fermentation medium to produce the C3-C6 alcohol; increasing the activity of the C3-C6 alcohol in a portion of the fermentation medium; distilling the portion of the fermentation medium to form a vapor phase comprising water and the C3-C6 alcohol and a liquid phase; condensing the vapor phase by contacting it with a solution comprising the C3-C6 alcohol, and conducting the liquid phase to the fermentation medium.

In another embodiment, the invention provides a method to recover a C3-C6 alcohol from a dilute aqueous solution that comprises a first amount of the C3-C6 alcohol, comprising distilling a portion of the dilute aqueous solution to form a vapor phase comprising the C3-C6 alcohol and water, wherein the vapor phase comprises between about 1% by weight and about 45% by weight of the first amount of C3-C6 alcohol from the portion of the dilute aqueous solution; and condensing the vapor phase by contacting with a solution comprising the C3-C6 alcohol.

The methods can further include spraying the solution comprising the C3-C6 alcohol into the vapor comprising the C3-C6 alcohol.

In some of embodiments of the methods, the solution comprising the C3-C6 alcohol comprises the condensate of the C3-C6 alcohol.

In some of embodiments of the methods, the condensate is cooled prior to being contacted with the C3-C6 alcohol vapor.

In other of embodiments of the methods, the step of forming the vapor or vapor phase and the step of condensing the vapor or vapor phase are conducted in a single vessel.

In other of embodiments of the methods, the vessel comprises a weir defining first and second fluid containing portions, wherein the first fluid containing portion is adapted to receive the aqueous solution or the fermentation medium comprising microorganisms and the C3-C6 alcohol, and the second fluid containing portion is adapted to receive the condensed vapor. In some embodiments, the first fluid containing portion comprises a conduit for conducting the aqueous solution or the fermentation medium comprising microorganisms and the C3-C6 alcohol into the first fluid containing portion and a conduit for conducting the aqueous solution or the fermentation medium comprising microorganisms and the C3-C6 alcohol out of the first fluid containing portion, wherein the content of the C3-C6 alcohol in the aqueous solution or the fermentation medium that is conducted out of the first fluid containing portion is less than that of the aqueous solution or the fermentation medium that is conducted into the first fluid containing portion.

In still other embodiments, the second fluid containing portion comprises a conduit for conducting the condensed vapor out of the second fluid containing portion.

In another embodiment, the invention provides a flash tank/direct contact condenser system for increasing the concentration of a C3-C6 alcohol in an aqueous solution comprising a vessel; means for introducing a stream of aqueous solution comprising the C3-C6 alcohol into the vessel; means for subjecting the stream of aqueous solution comprising the C3-C6 alcohol to reduced pressure to form a vapor comprising the C3-C6 alcohol; means for contacting the vapor comprising the C3-C6 alcohol with a solution comprising the C3-C6 alcohol to form a condensate comprising condensed vapor of the C3-C6 alcohol, wherein the concentration of the C3-C6 alcohol in the condensate is greater than the concentration of the C3-C6 alcohol in the first stream of aqueous solution.

In some embodiments, the vessel comprises two fluid containing compartments or portions that are separated by a weir, wherein the weir divides the compartments or portions at the bottom of the vessel.

In some embodiments, the means for subjecting the stream of aqueous solution comprising the C3-C6 alcohol to reduced pressure comprises a means for creating a vacuum.

In some embodiments, the means for contacting the vapor comprising the C3-C6 alcohol with a solution comprising the C3-C6 alcohol to form a condensate comprises a spray nozzle.

In another embodiment, the invention provides a method to recover a C3-C6 alcohol from a fermentation medium comprising microorganisms and the C3-C6 alcohol, comprising introducing a gas into the fermentation medium, wherein a portion of the C3-C6 alcohol transfers into the gas; conducting the gas from the fermentation medium to a recovery unit; and recovering the C3-C6 alcohol from the gas.

In some embodiments, the method further comprises increasing the activity of the C3-C6 alcohol in a portion of the fermentation medium to at least that of saturation of the C3-C6 alcohol in the portion, or decreasing the activity of water in a portion of the fermentation medium to at least that of saturation of the C3-C6 alcohol in the portion; forming a C3-C6 alcohol-rich liquid phase and a water-rich liquid phase from the portion of the fermentation medium; and separating the C3-C6 alcohol-rich phase from the water-rich phase.

In some embodiments, the method further comprises culturing a microorganism in a fermentation medium to produce the C3-C6 alcohol; and conducting the water rich phase to the fermentation medium.

In other embodiments, the method further comprises hydrolyzing a feedstock comprising a polysaccharide and at least one other compound to produce fermentable hydrolysis products; fermenting at least a portion of the fermentable hydrolysis products in a fermentation medium to produce the C3-C6 alcohol, wherein the fermentation medium further comprises at least one non-fermented compound; and separating the at least one non-fermented compound from the fermentation medium, the water-rich phase or both.

In some embodiments, the method further comprises distilling a vapor phase comprising water and the C3-C6 alcohol; and reacting the C3-C6 alcohol in the vapor phase to form a product.

In other embodiments, the method further comprises culturing a microorganism in a fermentation medium to produce the C3-C6 alcohol; increasing the activity of the C3-C6 alcohol in a portion of the fermentation medium; distilling the portion of the fermentation medium to produce a vapor phase comprising water and the C3-C6 alcohol, and a liquid phase, and conducting the liquid phase to the fermentation medium.

In still other embodiments, the method further comprises distilling a portion of the dilute aqueous solution to a vapor phase comprising C3-C6 alcohol and water, wherein the vapor phase comprises between about 1% by weight and about 45% by weight of the first amount of C3-C6 alcohol from the portion of the dilute aqueous solution; and condensing the vapor phase.

A method to operate a retrofit ethanol production plant comprising a pretreatment unit, multiple fermentation units, and a beer still to produce a C3-C6 alcohol, comprising pretreating a feedstock to form fermentable sugars in the pretreatment unit; culturing a microorganism in a fermentation medium comprising the fermentable sugars in a fermentation unit to produce the C3-C6 alcohol; introducing a gas into the fermentation medium, wherein a portion of the C3-C6 alcohol transfers into the gas; conducting the gas from the fermentation medium to a recovery unit; recovering the C3-C6 alcohol from the gas; treating a portion of the fermentation medium comprising the C3-C6 alcohol to remove a portion of the C3-C6 alcohol; returning the treated portion of the fermentation medium to the fermentation unit; and transferring the fermentation medium from the fermentation unit to the beer still.

In some embodiments at least about 50%, at least about 60%, at least about 70%, at least about 80%, at least about 85%, at least about 90%, or at least about 95% of the C3-C6 alcohol can be recovered from the gas.

In one embodiment, the invention provides a method for producing a C3-C6 alcohol comprising culturing a microorganism in a fermentation medium to grow the microorganism; culturing the microorganism in the fermentation medium to produce the C3-C6 alcohol; recovering the C3-C6 alcohol from the fermentation medium during the steps of culturing; and introducing a gas comprising oxygen into the fermentation medium during step producing the C3-C6 alcohol at an oxygen transfer rate (OTR) of less than about 20 mmoles of oxygen per liter of fermentation medium per hour.

In some embodiments, step of introducing comprises introducing a gas comprising oxygen into the fermentation medium during the step of producing at an OTR of less than about 10 mmoles of oxygen per liter of fermentation medium per hour, and in other embodiments, the step of introducing further comprises introducing a gas comprising oxygen into the fermentation medium at an OTR greater than the level required for the production of the C3-C6 alcohol, such as between about 0.5 and about 5 mmoles of oxygen per liter of fermentation medium per hour.

In some embodiments, the step of recovering the C3-C6 alcohol from the fermentation medium comprises the steps of increasing the activity of the C3-C6 alcohol in a portion of the fermentation medium to at least that of saturation of the C3-C6 alcohol in the portion, or decreasing the activity of water in a portion of the fermentation medium to at least that of saturation of the C3-C6 alcohol in the portion forming a C3-C6 alcohol-rich liquid phase and a water-rich liquid phase from the portion of the fermentation medium; and separating the C3-C6 alcohol-rich phase from the water-rich phase.

In some embodiments, the method further comprises the step of conducting the water rich phase to the fermentation medium.

In some embodiments, the method further comprises the steps of distilling a vapor phase comprising water and C3-C6 alcohol from the fermentation medium; and reacting the C3-C6 alcohol in the vapor phase to form a product.

In another embodiment, the invention provides a method to produce a C3-C6 alcohol, comprising culturing a microorganism in a fermentation medium to produce the C3-C6 alcohol; introducing a gas comprising oxygen into the fermentation medium during step of producing at an oxygen transfer rate (OTR) of less than about 20 mmoles of oxygen per liter of fermentation medium per hour; increasing the activity of the C3-C6 alcohol in a portion of the fermentation medium; distilling the portion of the fermentation medium to produce a vapor phase comprising water and C3-C6 alcohol and a liquid phase, and conducting the liquid phase to the fermentation medium.

In another embodiment, the invention provides a method to operate a retrofit ethanol production plant comprising a pretreatment unit, multiple fermentation units, and a beer still to produce a C3-C6 alcohol, comprising: pretreating a feedstock to form fermentable sugars in the pretreatment unit; culturing a microorganism in a fermentation medium comprising the fermentable sugars in a first fermentation unit to grow the microorganism; culturing the microorganism in the fermentation medium comprising the fermentable sugars in a first fermentation unit to produce the C3-C6 alcohol; introducing a gas comprising oxygen into the fermentation medium during step of producing at an oxygen transfer rate (OTR) of less than about 20 mmoles of oxygen per liter of fermentation medium per hour; treating a portion of the fermentation medium comprising the C3-C6 alcohol to remove a portion of the C3-C6 alcohol; returning the treated portion of the fermentation medium to the fermentation unit; and transferring the fermentation medium from the fermentation unit to the beer still.

In some embodiments of the methods the step of producing the C3-C6 alcohol is anaerobic.

In another embodiment, the invention provides a method for operating a process for production and recovery of a C3-C6 alcohol comprising multiple unit operations that are operated at less than atmospheric pressure, comprising the steps of introducing steam into a first eductor to create less than atmospheric pressure at a first unit operation; and conducting steam from the first eductor to a second eductor to create less than atmospheric pressure at a second unit operation.

In some embodiments, the multiple unit operations comprise unit operations selected from the group consisting of: a water reclamation, a first effect evaporator, a second effect evaporator, a beer still, side stripper and a rectifier.

In some embodiments, the first and second unit operations are the same and in other embodiments, the first and second unit operations are different.

In another embodiment, the invention provides a method to culture C3-C6 alcohol producing microorganisms to high cell densities comprising the steps of growing the microorganisms in a fermentation medium and recovering the C3-C6 alcohol from the fermentation medium during the step of growing; wherein the microorganisms reach a cell density ranging from about 5 g per liter to about 150 g per liter dry weight.

In another embodiment, the invention provides a method to produce a C3-C6 alcohol comprising the steps of culturing microorganisms that produce the C3-C6 alcohol in a fermentation medium to produce the C3-C6 alcohol and recovering the C3-C6 alcohol from the fermentation medium; wherein the production of the C3-C6 alcohol is at a rate of at least about 1 g per liter per hour.

In some embodiments, the production of the C3-C6 alcohol is at a rate of at least about 2 g per liter per hour.

In some embodiments, the C3-C6 alcohol is a butanol and in other embodiments, the C3-C6 alcohol is isobutanol.

The invention also provides, in a further embodiment, a method to recover a C3-C6 alcohol from a dilute aqueous solution at a first temperature (T1) comprising distilling a vapor phase comprising water and C3-C6 alcohol from the dilute aqueous solution; condensing the vapor phase with an aqueous cooling fluid at a second temperature (T2); controlling the pressure of the step of distilling, T1 and the C3-C6 alcohol titer so that the temperature of the vapor phase is a third temperature (T3), wherein difference between T3 and T2 is at least about 1° C.

In some embodiments, the difference between T3 and T2 is at least about 5° C., and in other embodiments, the difference between T3 and T2 is at least about 10° C.

In some embodiments, T2 is less than about 30° C.

In other embodiments, the aqueous cooling fluid at a second temperature (T2) is produced by evaporative cooling.

In other embodiments, a portion of condensed vapor phase is used as the aqueous cooling fluid.

In some embodiments, the method further comprises forming a C3-C6 alcohol-rich liquid phase and a water-rich liquid phase from the condensed vapor phase.

In some embodiments, the method further comprises separating the C3-C6 alcohol-rich phase and the water-rich phase.

In other embodiments, the vapor phase comprises between about 2% by weight and about 40% by weight of the C3-C6 alcohol from the dilute aqueous solution.

In some embodiments, the step of distilling is adiabatic and in other embodiments the step of distilling is isothermal.

In some embodiments the dilute aqueous solution comprises a fermentation medium comprising a microorganism, the method further comprising culturing the microorganism in the fermentation medium to produce the C3-C6 alcohol; and conducting the water rich phase to the fermentation medium.

### BRIEF DESCRIPTION OF THE DRAWINGS

FIG. 1 represents an embodiment of the present invention for the production and recovery of iso-butanol.

FIG. 2 represents an embodiment of the present invention for the production and recovery of butanol from fermentation broth in a process of simultaneous saccharification and fermentation of pretreated corn.

FIG. 3 represents an embodiment of the present invention for the production and recovery of a C3-C6 alcohol from fermentation broth using a gas scalper.

FIG. 4 represents an embodiment of a flash tank/direct contact condenser unit.

FIG. 5 represents an embodiment of the present invention for the production and recovery of a C3-C6 alcohol from fermentation broth using a flash tank/direct contact condenser unit.

FIG. 6 represents an embodiment of the present invention for the production and recovery of a C3-C6 alcohol from fermentation broth using a gas stripper.

FIG. 7 represents an embodiment of the present invention for the production and recovery of a C3-C6 alcohol from fermentation broth using aeration.

FIG. 8 represents an embodiment of the present invention for the production and recovery of a C3-C6 alcohol from fermentation broth using a flash tank/direct contact condenser unit and a gas scalper.

FIG. 9 represents an embodiment of the present invention for the production and recovery of a C3-C6 alcohol from fermentation broth using a flash tank/direct contact condenser unit and gas stripper.

FIG. 10 provides a comparison of isobutanol broth titer in the fermentor (closed marker) and remaining isobutanol titer in the broth after the flash tank (open marker),

FIG. 11 shows the effective isobutanol titer in g/L and gallons and volumetric productivity in a 10,000 liter production fermentor. Isobutanol was calculated from the amount of glucose consumed at 90% theoretical yield.

FIG. 12 represents a process flow for purification of isobutanol by distillation using a two column system.

FIG. 13 represents an embodiment of the present invention for the production and recovery of a C3-C6 alcohol from fermentation broth using a flash tank/direct contact condenser unit, a gas scalper and a three pump loop.

### DETAILED DESCRIPTION OF THE INVENTION

The present invention describes methods for recovery of C3-C6 alcohols from dilute aqueous solutions, such as fermentation broths, related systems, and methods. Related methods include, for example, methods to produce products from C3-C6 alcohols in dilute aqueous solutions. As used herein the term C3-C6 alcohol refers to an alcohol containing three, four, five or six carbon atoms, including all of the isomers thereof, and mixtures of any of the foregoing. Thus, the C3-C6 alcohol can be selected from propanols, butanols, pentanols, and hexanols. More particularly, the C3 alcohol may be 1-propanol, or 2-propanol; the C4 alcohol may be 1-butanol, 2-butanol, tert-butanol (2-methyl-2-propanol), or iso-butanol (2-methyl-1-propanol); the C5 alcohol may be 1-pentanol, 2-pentanol, 3-pentanol, 2-methyl-1-butanol, 3-methyl-1-butanol, 2-methyl-2-butanol, 3-methyl-2-butanol, or 2,2-dimethyl-1-propanol; and the C6 alcohol may be 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 3,3-dimethyl-1-butanol, 2,2-dimethyl-1-butanol, 2,3-dimethyl-1-butanol, 2,3-dimethyl-2-butanol, 3,3-dimethyl-2-butanol, or 2 ethyl-1-butanol. In a preferred embodiment, the C3-C6 alcohol is iso-butanol (2-methyl-1-propanol). In some embodiments, the ratio of the C3-C6 alcohol to water in the dilute aqueous solution is less than about 10/90 (w/w), less than about 9/91 (w/w), less than about 8/92 (w/w), less than about 7/93 (w/w), less than about 6/94 (w/w), less than about 5/95 (w/w), less than about 4/96 (w/w), less than about 3/94 (w/w), less than about 2.5/97.5 (w/w), less than about 2/98 (w/w), less than about 1.5/98.5 (w/w), less than about 1/99 (w/w), or less than about 0.5/99.5 (w/w). A “dilute” aqueous solution as used herein can mean a solution containing the C3-C6 alcohol at a concentration below the solubility limit of the C3-C6 alcohol in the solution. Concentration can be expressed in a variety of different units, e.g. weight or volume percent, molar concentration, molal concentration or alcohol/water w/w of v/v ratio. Unless specified otherwise, however, the concentrations are generally presented here as weight percent. In case of a stream comprising at least one additional compound (e.g. solute, solvent, adsorbent, etc.), alcohol weight concentration as used herein is calculated by 100 times alcohol weight in that stream divided by the combined weights of alcohol and water in that stream.

In some embodiments, the methods of the present invention include the step of gas scalping (or gas removal) from a fermentation broth or a dilute aqueous solution prior to recovery of a C3-C6 alcohol or production of products from C3-C6 alcohols. Gas scalping is used to remove CO₂ and other gases. The gases present in a fermentation broth or a dilute aqueous solution may include any gas that is present in the air or that is produced during fermentation. Examples of such gases include, without limitation, carbon-dioxide, oxygen and nitrogen. The removal of gases can be effected by employing any known process. For example, gases can be removed by heating, applying reduced pressure and pulling a partial vacuum, adding suitable adsorbents to adsorb the gases, or a combination of these processes. In a preferred embodiment, gas scalping is performed in a stream comprising a C3-C6 alcohol prior to introducing the stream to a flash tank, distillation operation or any subsequent treatment involving volatilization of the alcohol, discussed in detail below.

Gas scalping prior to such subsequent treatment allows for a number of advantages. When alcohol is recovered from a stream by use of a flash tank, distillation operation or other similar treatment, if the stream also includes a gas or gases, such as carbon dioxide, any gases in the stream will be volatilized as well and become part of the vapor. Volatilization of gas along with the alcohol has the significant disadvantage of increasing the volume of the vapor comprising the alcohol. The equipment and process requirements for handling a larger volume and the associated energy costs significantly increase the cost of such an operation. In contrast, by selectively removing the gas, prior to volatilizing the alcohol, the volume of the vapor containing the alcohol is smaller and can be handled more efficiently. For example, in an embodiment, as discussed below, in which a deep vacuum is pulled on a flash tank by use of steam eductors in series, the volume of non-condensable species in the flash tank exiting through the eductors is greatly reduced with prior scalping of gases. Gas scalping can be used in various embodiments contemplated in the invention, such as the following embodiments.

For example, in one embodiment, the present invention includes a method to recover a C3-C6 alcohol from a dilute aqueous solution of the C3-C6 alcohol, such as a fermentation broth comprising microorganisms, gas and the C3-C6 alcohol. This method includes removing at least a portion of the gas from the aqueous solution and increasing the activity of the C3-C6 alcohol in the portion of the aqueous solution to at least that of saturation of the C3-C6 alcohol in the portion, or similarly, decreasing the activity of water in the portion of the fermentation medium to at least that of saturation of the C3-C6 alcohol in the portion. The method further includes forming a C3-C6 alcohol-rich liquid phase and a water-rich liquid phase from the portion of the aqueous solution, and separating the C3-C6 alcohol-rich phase from the water-rich phase. This embodiment can also include culturing a microorganism in the fermentation medium to produce the C3-C6 alcohol and gases, conducting at least a portion of the water rich phase to the fermentation medium and optionally, distilling the portion of a fermentation medium to produce a vapor phase comprising water and C3-C6 alcohol and a liquid phase. It should be recognized that reference to conducting at least a portion of the water rich phase to the fermentation medium can mean either conducting the water rich phase itself to the fermentation medium or more often, treating the water rich phase, for example, to recover more alcohol from it and then conducting some remaining portion of the water rich phase to the fermentation medium. For example, if the water rich phase has a higher concentration of alcohol than does the fermentation medium, it is unlikely to be beneficial to introduce it to the fermentation medium. Typically in such a case, the water rich fraction will be further processed, such as in a beer still to recover more alcohol, before a portion of the water rich phase is conducted to the fermentation medium. Alternatively, this embodiment can include hydrolyzing a feedstock comprising a polysaccharide and at least one other compound to produce fermentable hydrolysis products, fermenting at least a portion of the fermentable hydrolysis products in the fermentation medium to produce the C3-C6 alcohol and gases, wherein the fermentation medium further comprises at least one non-fermented compound, and separating the non-fermented compound from the fermentation medium, or the water-rich phase, or both.

In another embodiment, the invention provides a method to produce a product from a C3-C6 alcohol in a fermentation medium comprising microorganisms, gas and the C3-C6 alcohol. This method includes removing at least a portion of the gas from the fermentation medium; distilling a vapor phase comprising water and C3-C6 alcohol from the fermentation medium; and reacting the C3-C6 alcohol in the vapor phase to form the product.

In still another embodiment, the invention provides a method to recover a C3-C6 alcohol from a dilute aqueous solution that comprises a first amount of the C3-C6 alcohol and gas. This method includes removing at least a portion of the gas from the dilute aqueous solution and distilling a portion of the dilute aqueous solution to a vapor phase comprising C3-C6 alcohol and water, wherein the vapor phase comprises between about 1% by weight and about 45% by weight of the first amount of C3-C6 alcohol from the portion of the dilute aqueous solution; and condensing the vapor phase. In various alternative embodiments, the vapor phase can comprise between about 2% by weight and about 40% by weight of the C3-C6 alcohol, between about 3% by weight and about 35% by weight of the C3-C6 alcohol and between about 4% by weight and about 30% by weight of the C3-C6 alcohol and between about 5% by weight and about 25% by weight of the C3-C6 alcohol present in the portion of the dilute aqueous solution. By controlling or limiting the amount of alcohol in the solution that is distilled to the vapor phase, a number of important advantages are achieved, as discussed, for example in WO 2009/086391 A2, which is hereby incorporated by reference in its entirety.

A still further embodiment involving gas scalping is a process to operate a retrofit ethanol production plant comprising a pretreatment unit, multiple fermentation units, and a beer still to produce a C3-C6 alcohol. This process includes pretreating a feedstock to form fermentable sugars in the pretreatment unit and culturing a microorganism in a fermentation medium comprising the fermentable sugars and gas in a first fermentation unit to produce the C3-C6 alcohol. The process further includes removing at least a portion of the gas from the fermentation medium, treating a portion of the fermentation medium comprising the C3-C6 alcohol to remove a portion of the C3-C6 alcohol, returning the treated portion of the fermentation medium to the first fermentation unit, and transferring the fermentation medium from the first fermentation unit to the beer still.

In embodiments of the present invention where the gas scalping is used, while there can be other gases, as noted above, carbon dioxide is a primary concern because it is typically, the largest component of gases dissolved in a fermentation broth. Therefore, in various embodiments, at least about 30% of the carbon dioxide is removed during the step of removing at least a portion of gas from a dilute aqueous solution or fermentation broth, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

As noted above, gas removal (or scalping) can be effected by any suitable method, such as heating the aqueous stream to volatilize the gas, reducing pressure on the stream to below atmospheric pressure to volatilize the gas, adsorption of the gas from the aqueous stream and combinations thereof. In embodiments in which the step of removing includes heating the aqueous stream to volatilize the gas, suitable volatilization temperatures depend on the pressure on the stream, as well as the particular gas or gases being removed and the temperature at which the alcohol will remain in solution without volatilizing. More particularly, suitable temperatures can be between about 20° C. and about 95° C., between about 25° C. and about 55° C., or between about 30° C. and about 50° C. In embodiments in which the step of removing includes reducing pressure to volatilize the gas, the pressure can be reduced to a pressure of between about 1 psia and about 10 psia, between about 1 psia and about 8 psia, between about 3 psia and about 10 psia, or between about 2 psia and about 5 psia.

Once removed, the scalped gas (comprising carbon dioxide or other gases) can be vented or integrated into the overall process. For example, in the instance in which the gas is or comprises carbon dioxide, the carbon dioxide can be conducted to a fermentation unit for pH control. Alternatively, carbon dioxide can be compressed to make dry ice. In addition, the removed gas may also include some amount of C3-C6 alcohol volatilized along with the gas even though the majority of the C3-C6 alcohol is intended to remain in the aqueous stream. In such an instance, the removed gas can be treated to remove the C3-C6 alcohol from the gas. For example, C3-C6 alcohol can be recovered by the use of a water scrubber, pressurization and condensation, or adsorption (e.g., with carbon).

The fermentation broth or dilute aqueous solution, in addition to containing a C3-C6 alcohol and one or more gases, can contain other impurities. Thus, in some embodiments, the methods further include removing at least one impurity from the fermentation medium or the dilute aqueous solution. The term “impurity” or “impurities” means any compound other than water and the alcohol being purified. The term impurity includes any byproduct or co-product of the fermentation process i.e. a product related to the production of alcohol, other than the alcohol, in any amount or in an undesired amount. In some embodiments, the impurity can be selected from ethanol, acetic acid, propanol, phenyl ethyl alcohol, isopentanol or combinations of these impurities. Removal of impurities can be effected by any suitable method, such as heating the aqueous stream to volatilize the impurity, reducing pressure on the stream to below atmospheric pressure to volatilize the impurity, or combinations thereof. In embodiments in which the step of removing includes heating the aqueous stream to volatilize the impurity, suitable volatilization temperatures depend on the pressure on the stream, as well as the particular impurity or impurities being removed and the temperature at which the alcohol will remain in solution without volatilizing. More particularly, suitable temperatures can be between about 20° C. and about 95° C., between about 25° C. and about 55° C., or between about 30° C. and about 50° C. In embodiments in which the step of removing includes reducing pressure to volatilize the impurity, the pressure can be reduced to a pressure of between about 1 psia and about 10 psia, between about 1 psia and about 8 psia, between about 3 psia and about 10 psia, or between about 2 psia and about 5 psia. Reference herein to purification or removing impurities means increasing the ratio between a product and another compound other than water.

Removal of impurities beneficially occurs prior to increasing activity of the alcohol, decreasing activity of the water or distilling for recovery of the alcohol. The removal of impurities may be performed during the same operation in which the gases are removed or after such an operation. In the instance of using increased temperature, reduced pressure or a combination, typically gases such as carbon dioxide and nitrogen will be removed first. Depending upon the relative volatility of the impurity and alcohol product, the impurity will be removed next i.e. after the gases come off but before any significant removal of the C3-C6 alcohol takes place. Relative volatility is a function of the activity coefficient, molecular concentration and vapor pressure saturation. It may be that at this step, some C3-C6 alcohol is lost along with the impurity. However, it is possible to recover the C3-C6 alcohol from this stream.

Removal of impurities prior to subsequent treatment for recovery of the alcohol product allows for a number of advantages. When alcohol is recovered from a stream by use of a flash tank, distillation operation or other similar treatment, if the stream also includes a volatile impurity that will be vaporized with the alcohol, such as acetic acid, any such impurities in the stream will be volatilized as well and become part of the vapor. Volatilization of impurities along with the alcohol has the significant disadvantage of increasing the volume of the vapor comprising the alcohol. The equipment and process requirements for handling a larger volume and the associated energy costs significantly increase the cost of such an operation. In contrast, by selectively removing the impurities, prior to volatilizing the alcohol, the volume of the vapor containing the alcohol is smaller and can be handled more efficiently.

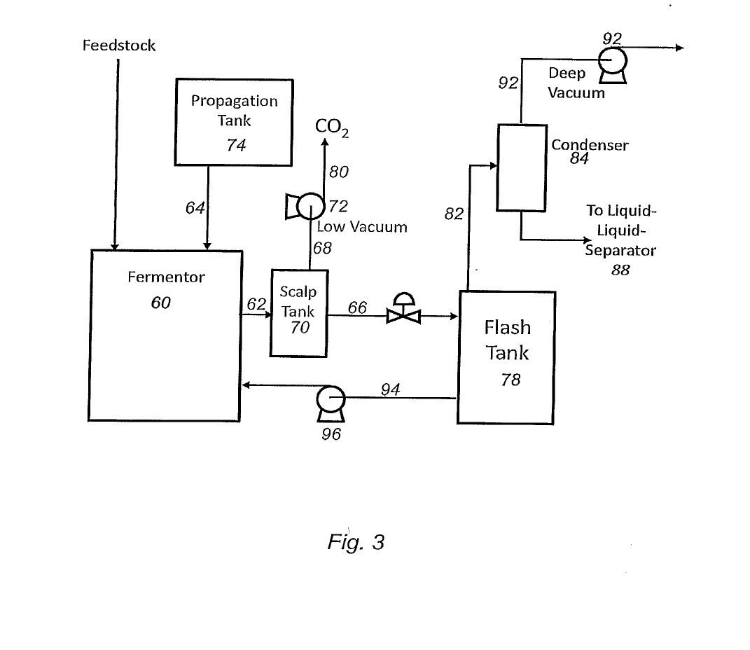

With reference to FIG. 3, an embodiment of the present invention illustrating the use of scalping is shown. Fermentation is conducted in fermentor 60. The fermentation broth in the fermentor 60 includes the C3-C6 alcohol product, and other components of the fermentation medium. During the course of the fermentation, a stream of the fermentation broth, which may include microorganisms, is conducted from the fermentor 60 to a scalp tank 70 via 62. The scalper can be operated at a pressure of about 1 to about 10 psia. Under these conditions it is primarily the dissolved gases that are removed from the fermentation broth while the C3-C6 alcohols remain in the broth. Because the dissolved gases are removed prior to the flash, they do not form part of the flash vapor traffic and thus are not processed with the C3-C6 alcohol recovery system. Removal of gases from the scalp tank is effected by pulling a partial vacuum by vacuum pump 72 via 68 to a vent stream 80. A propagation tank 74 conducts an initial culture to the fermentor 60 via 64. After the scalp tank removes gases from the fermentation broth, the broth is further conducted to a flash tank 78 for distillation via 66. The fermentation heat can partially supply the heat required for vaporization in the flash system. The flash tank 78 is maintained at below atmospheric pressure so that upon introduction of the degassed fermentation broth into the flash tank 78, a portion of the fermentation broth gets vaporized. The portion of the vaporized fermentation broth includes only a portion of the alcohol in the fermentation broth along with Water vapor. After distillation in the flash tank 78, the remaining portion of the fermentation broth that is not distilled is returned to the fermentor 60 via 94 and pump 96. This fermentation broth that is being returned to the fermentor is now partially depleted of alcohol. The portion of the fermentation broth that is vaporized in the flash tank 78 is conducted as a vapor to a vapor condenser 84 via 82. Upon condensation of the mixed alcohol and water vapor, the condensed solution is conducted to a liquid-liquid separator 88 via 86. The remaining vapor that is not condensed is then further conducted to an outlet via 90 and 92.

In some embodiments, methods of the present invention are directed to increasing the concentration of a C3-C6 alcohol in an aqueous solution, recovering a C3-C6 alcohol from a fermentation medium or dilute aqueous solution, or producing a C3-C6 alcohol which includes forming a vapor phase containing the C3-C6 alcohol and contacting the vapor with a solution comprising the C3-C6 alcohol to condense the vapor phase. A significant advantage of such methods is that by directly contacting a vapor with a condensing solution (as compared to indirect contact in a shell and tube condenser), the difference in temperature between the vapor and the condensing solution can be relatively small and still effectively condense the vapor. Thus, the energy requirements for cooling the condensing solution are less, resulting in more energy efficient processes. A further significant advantage of such methods, particularly when the C3-C6 alcohol content of the condensing solution is approximately the same as the vapor when condensed, is that the condensing solution and the condensed vapor can be comingled without significantly diluting the alcohol content of either one. In these embodiments, the aqueous solution may be subjected to reduced pressure and/or increased temperature to volatilize the alcohol and form a vapor. For example, the aqueous solution may be heated prior to being conducted into a flash tank, for example by utilizing a heat exchanger, or may be heated inside the flash tank, for example by utilizing heating coils.

For example, in one embodiment, the invention provides a method for increasing the concentration of a C3-C6 alcohol in an aqueous solution. This method includes introducing a first stream of aqueous solution containing the C3-C6 alcohol into a vessel; subjecting the first stream to reduced pressure to form a vapor containing the C3-C6 alcohol; contacting the vapor with a solution containing the C3-C6 alcohol to form a condensate, wherein the concentration of the C3-C6 alcohol in the condensate is greater than the concentration of the C3-C6 alcohol in the first stream of aqueous solution.

In another embodiment, the invention provides a method to recover a C3-C6 alcohol from a fermentation medium containing microorganisms and the C3-C6 alcohol. This method includes increasing the activity of the C3-C6 alcohol in a portion of the fermentation medium to at least that of saturation of the C3-C6 alcohol in the portion to form a vapor including the C3-C6 alcohol, or decreasing the activity of water in a portion of the fermentation medium to at least that of saturation of the C3-C6 alcohol in the portion to form a vapor containing the C3-C6 alcohol. The C3-C6 alcohol vapor is condensed by contacting the vapor containing the C3-C6 alcohol with a solution containing the C3-C6 alcohol. The condensate loins a C3-C6 alcohol-rich liquid phase and a water-rich liquid phase from the condensed vapor, and the method further includes separating the C3-C6 alcohol-rich phase from the water-rich phase. This method can further include culturing a microorganism in the fermentation medium to produce the C3-C6 alcohol; and conducting at least a portion of the water rich phase to the fermentation medium. Other embodiments relating to fermentation processes are contemplated, such as those that further include the step of hydrolyzing a feedstock containing a polysaccharide, which is described elsewhere herein.

In still another embodiment, the invention provides a method to produce a C3-C6 alcohol by culturing a microorganism in a fermentation medium to produce the C3-C6 alcohol. This method further includes increasing the activity of the C3-C6 alcohol in a portion of the fermentation medium and distilling the portion of the fermentation medium to form a vapor phase including water and the C3-C6 alcohol and a liquid phase. The vapor phase is condensed by contacting it with a solution containing the C3-C6 alcohol, and conducting the liquid phase to the fermentation medium.

A still further embodiment involving contacting a vapor with a solution comprising the C3-C6 alcohol to condense the vapor phase is a method to recover a C3-C6 alcohol from a dilute aqueous solution that contains a first amount of the C3-C6 alcohol, by distilling a portion of the dilute aqueous solution to form a vapor phase containing the C3-C6 alcohol and water, wherein the vapor phase comprises between about 1% by weight and about 45% by weight of the first amount of C3-C6 alcohol from the portion of the dilute aqueous solution. This method further includes condensing the vapor phase by contacting with a solution containing the C3-C6 alcohol.

In embodiments of the present invention that include forming a vapor phase containing the C3-C6 alcohol, and contacting the vapor with a solution comprising the C3-C6 alcohol, the step of contacting can include spraying a solution containing the C3-C6 alcohol into the vapor containing the C3-C6 alcohol. In other embodiments, the solution containing the C3-C6 alcohol can be or include the condensate of the C3-C6 alcohol from the vapor phase. That is, as the vapor is condensed to form a solution, a portion of that solution can be used as the solution comprising C3-C6 alcohol to condense additional vapor. In this manner, the concentration of C3-C6 alcohol in the solution and in the condensed vapor is similar if not the same and there are no concerns about diluting the concentration of the C3-C6 alcohol.

In embodiments in which the vapor is contacted with a solution containing the C3-C6 alcohol that includes condensate of the C3-C6 alcohol from the vapor phase, the solution can be cooled prior to contact with the C3-C6 alcohol vapor. The condensate may be cooled using any conventional cooling process, for example, using a heat exchanger. Any cooling fluid used in such a heat exchanger can be cooled using processes, such as chilling or as discussed below, evaporative cooling.

The step of forming the vapor or vapor phase and the step of condensing the vapor or vapor phase can be conducted in a single vessel. Such a vessel can include a weir (a partial barrier that divides compartments or portions at the bottom of the vessel) defining first and second fluid containing portions of the vessel. The two fluid containing compartments or portions are open at the top of the vessel and communicate with each other, maintaining separation of the fluids but allowing for movement of vapor. In this embodiment, the first fluid containing portion will receive the aqueous solution or the fermentation medium comprising microorganisms and the C3-C6 alcohol, and the second fluid containing portion is will receive the condensed vapor.

In some embodiments, the first fluid containing portion of the vessel includes a conduit for conducting the aqueous solution or the fermentation medium comprising microorganisms and the C3-C6 alcohol into the first fluid containing portion and a conduit for conducting the aqueous solution or the fermentation medium comprising microorganisms and the C3-C6 alcohol out of the first fluid containing portion. The content of the C3-C6 alcohol in the aqueous solution or the fermentation medium that is conducted out of the first fluid containing portion is less than that of the aqueous solution or the fermentation medium that is conducted into the first fluid containing portion. In other embodiments, the second fluid containing portion comprises a conduit for conducting the condensed vapor out of the second fluid containing portion.

A further embodiment of the present invention that includes forming a vapor phase containing the C3-C6 alcohol and contacting the vapor with a solution comprising the C3-C6 alcohol to condense the vapor phase is a method to recover a C3-C6 alcohol from a dilute aqueous solution at a first temperature (T1) that includes distilling a vapor phase comprising water and C3-C6 alcohol from the dilute aqueous solution. The process further includes condensing the vapor phase with an aqueous cooling fluid at a second temperature (T2) and controlling the pressure of the step of distilling, T1 and the C3-C6 alcohol titer so that the temperature of the vapor phase is a third temperature (T3), wherein difference between T3 and T2 is at least about 1° C. In some embodiments of this method, the difference between T3 and T2 is at least about 2° C., about 3° C., about 4° C., about 5° C., about 6° C., about 7° C., about 8° C., about 9° C., about 10° C., about 11° C., about 12° C., about 13° C., about 4° C. or about 15° C. In other embodiments, T2 is less than about 30° C., about 29° C., about 28° C., about 27° C., about 26° C., about 25° C., about 24° C., about 23° C., about 22° C., about 21° C., about 20° C.

In other embodiments of this method, the aqueous cooling fluid at a second temperature (T2) is produced by evaporative cooling. Reference to being produced by evaporative cooling herein means that the temperature of a fluid in question has been modified or influenced by an evaporative cooling process. For example, in this embodiment the aqueous cooling fluid being produced by evaporative cooling can refer to the fluid being cooled, for example, by a heat exchanger in which the fluid that cools the aqueous cooling fluid is itself cooled by evaporative cooling. Evaporative cooling refers to lowering the temperature of a liquid by utilizing the latent heat of vaporization of a portion of the liquid. Significant advantages in the present process are achieved by the use of an aqueous cooling fluid produced by evaporative cooling. More particularly, the use of evaporative cooling, as opposed for example to cooling with a chiller that uses a compressor, is that evaporative cooling is significantly more energy efficient. By controlling the pressure of the step of distilling, T1 and the C3-C6 alcohol titer so that the temperature of the vapor phase is such that it can be condensed, with the aqueous cooling fluid at T2, produced by evaporative cooling, the process is more energy efficient than if the aqueous cooling fluid was produced by a more energy intensive process.

In still other embodiments of this method, a portion of condensed vapor phase can be used as the aqueous cooling fluid. In addition, this method can include further recovery steps. In particular, a C3-C6 alcohol-rich liquid phase and a water-rich liquid phase can be formed from the condensed vapor phase. The C3-C6 alcohol-rich phase and the water-rich phase can then be separated. Also, the step of distilling can be either adiabatic or isothermal. Further, in certain embodiments, the vapor phase includes between about 2% by weight and about 40% by weight of the C3-C6 alcohol from the dilute aqueous solution, particularly in the case of an adiabatic distillation. Further, in other embodiments, the vapor phase includes between about 2% by weight and about 90% by weight of the C3-C6 alcohol from the dilute aqueous solution, particularly in the case of an isothermal distillation. The dilute aqueous solution can be a fermentation medium comprising a microorganism, and the method can include culturing the microorganism in the fermentation medium to produce the C3-C6 alcohol; and conducting the water rich phase to the fermentation medium.

Further embodiments of the present invention include a system having dual function as a flash tank and a direct contact condenser of a vapor that functions for increasing the concentration of a C3-C6 alcohol in an aqueous solution. The system includes a vessel. The combination of these functions allows the formation of a deep vacuum sufficient to flash a C3-C6 alcohol-containing stream and recover alcohol while reducing capital and operating costs. To ensure a similar pressure drop with a separate flash tank and direct contact condenser would require relatively large connective piping involving significant expenditure. Thus, capital costs are reduced since the need for large connective infrastructure is avoided. In particular, one embodiment of the flash tank/direct contact condenser system for increasing the concentration of a C3-C6 alcohol in an aqueous solution includes a vessel; a conduit or other conveyance for introducing a stream of aqueous solution containing the C3-C6 alcohol into the vessel, a conduit or other conveyance for subjecting the stream of aqueous solution comprising the C3-C6 alcohol to reduced pressure to form a vapor comprising the C3-C6 alcohol; a conduit or other conveyance for contacting the vapor containing the C3-C6 alcohol with a solution containing the C3-C6 alcohol to form a condensate comprising condensed vapor of the C3-C6 alcohol, such that the concentration of the C3-C6 alcohol in the condensate is greater than the concentration of the C3-C6 alcohol in the first stream of aqueous solution.

Flash tank vacuum evaporation operations have less engineering concerns regarding pressure drop under vacuum because the flash tank acts as a single stage of separation without stages of liquid above the flash tank impacting pressure drop on the system, and the differential pressure across flash tank operations can be very low. Design calculations for vapor generation in the flash tank and sizing of piping systems can be appropriately selected to achieve low pressure drop. The distillation of a C3-C6 alcohol in a flash tank requires less vacuum than a distillation column and, thus, the flash tank has lower operating cost and capital costs inasmuch as the equipment is smaller in size and simpler in construction.

In any embodiments of the present invention involving a step of flashing a C3-C6 alcohol-containing solution, the flash can be done either adiabatically or isothermally. As noted above, the vapor phase from a flash operation can include between about 2% by weight and about 40% by weight of the C3-C6 alcohol from a dilute aqueous solution, particularly in the case of an adiabatic distillation. Further, in other embodiments, the vapor phase can include between about 2% by weight and about 90% by weight of the C3-C6 alcohol from a dilute aqueous solution, particularly in the case of an isothermal distillation. The use of an adiabatic flash has the advantage that the equipment for conducting such a process is simple and therefore, has relatively low capital cost. However, the amount of C3-C6 alcohol that can be removed under these conditions is practically limited as compared to the use of an isothermal process. Consequently, to meet the requirements of alcohol removal from the fermentor, the flow rate to/from a flash tank (and consequently, the turnover rate of the fermentor, expressed as 1/hr) operated adiabatically can be significantly greater than for a flash tank operated isothermally. Thus, isothermal operation of a flash tank has the significant advantage of allowing a lower flow rate between a flash tank and a fermentor resulting in the ability to use smaller and more standard equipment.

In embodiments of the present invention involving a flash operation, the turnover rate can be between about 0.033/hr and about 1/hr or between about 0.125/hr and about 0.25/hr. In embodiments of the present invention involving a flash operation and particularly an isothermal flash, the turnover rate can be between about 0.033/hr and about 0.33/hr or between about 03.04/hr and about 0.25/hr. In embodiments of the present invention involving a flash operation and particularly an adiabatic flash, the turnover rate can be between about 0.25/hr and about 1/hr or between about 0.25/hr and about 0.5/hr. It will be appreciated that the flow rate to/from a flash tank represented by these turnover rates is dependent on the volume of the fermentor.

A further advantage of an isothermal flash is that because it is operated at a constant temperature, the amount of alcohol in the vapor is greater than in an adiabatic operation in which the temperature drops during the flash. Therefore, when the vapor is condensed, the condensate is more enriched in alcohol and there is less water to handle as the alcohol is being recovered.

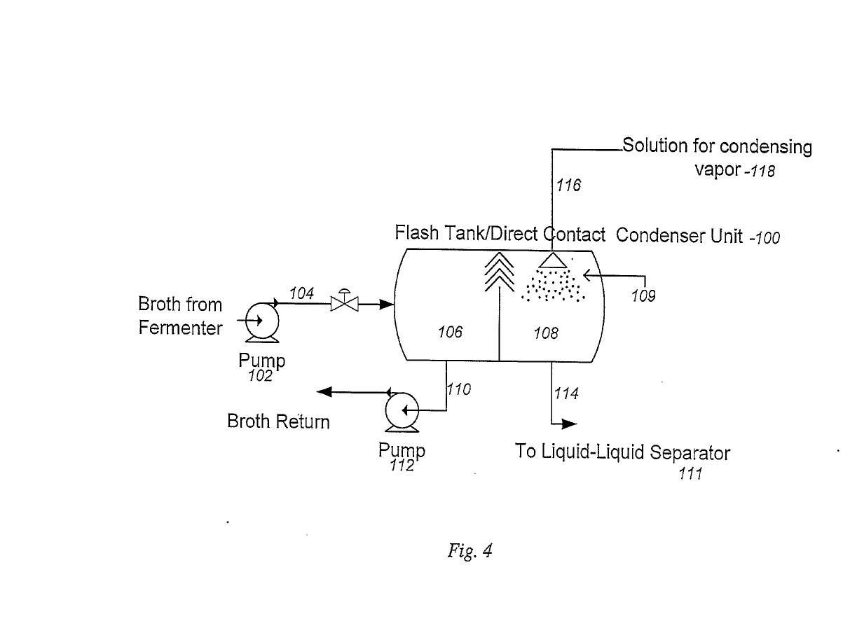

An embodiment of the flash tank/direct contact condenser unit is shown in FIG. 4. As shown, the unit comprises a vessel 100 which contains two fluid containing compartments 106, 108 or portions that are separated by a weir or partial barrier that divides the compartments 106, 108 or portions at the bottom of the vessel 100. Thus, the two fluid containing compartments 106, 108 or portions are open at the top of the vessel 100 and communicate with each other, maintaining separation of the fluids but allowing for movement of vapor. The flash tank/direct contact condenser unit is adapted to create a vacuum, such as with a mechanical vacuum device or an eductor vacuum device, so that the C3-C6 alcohol can be volatilized. The left or first fluid containing portion 106 is adapted to receive a dilute aqueous solution containing the C3-C6 alcohol via 104 and pump 102. Such solution may be a fermentation broth containing microorganisms and the C3-C6 alcohol. As such, this portion can comprise two conduits, one for introducing a stream of the dilute aqueous solution into this portion 106 via 104 and pump 102, for example a conduit or pipe, and the other for conducting the solution (partially depleted of alcohol) out of this portion after flashing and volatilizing the C3-C6 alcohol via 110 and pump 112. The right or second fluid containing portion 108 is adapted to receive a solution for condensing vapor comprising the C3-C6 alcohol 118. Although this solution may comprise water or any C3-C6 alcohol, in preferred embodiments it comprises the same C3-C6 alcohol that is being produced and/or recovered. The second portion 108 also comprises two conduits, one for introducing the solution comprising the C3-C6 alcohol into this portion 116 and another for conducting condensed vapor out of this portion 114, for example, to a liquid-liquid separator 111. The solution may be introduced by employing a spraying mechanism 109, such as a spray nozzle, spray ball or other mechanism suitable to condense vapor comprising C3-C6 alcohol.

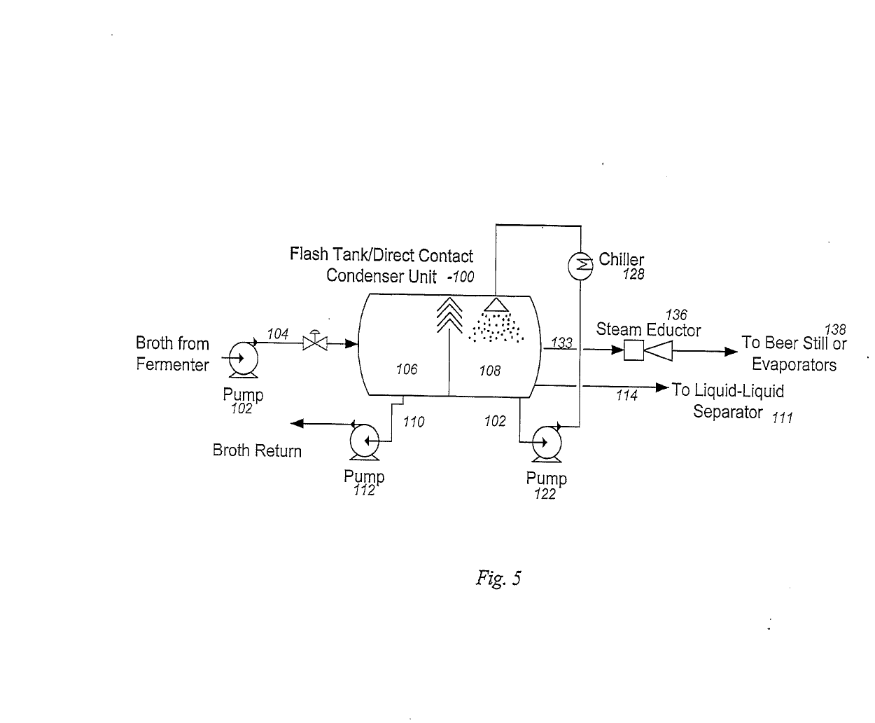

A particular embodiment of a flash tank/direct contact condenser unit 100 is shown in FIG. 5. In this embodiment, a stream of fermentation broth from a fermentor comprising microorganisms and a C3-C6 alcohol is introduced into the left or first portion of the flash tank/direct contact condenser unit 106 via 104 and pump 102. A portion of the fermentation broth is flashed by subjecting the broth to low pressure to form a vapor comprising the C3-C6 alcohol. The low pressure is created by a steam eductor 109. The stream 133 that is pulled by the eductor 136 can be sent for further processing and recovery of alcohol values to a beer still or evaporators 138. The remaining broth is returned to the fermentor via 110 and pump 112; and in the returning broth, the content of the C3-C6 alcohol is less than that in the initial stream of the broth. The vapor comprising the C3-C6 alcohol is contacted with a solution in the right or second portion of the unit 108 to condense the vapor to form a solution comprising the C3-C6 alcohol (the condensate). The content of the C3-C6 alcohol in the condensate is greater than that in the initial stream of the broth. The condensate may be conducted to a liquid-liquid separator 111 via 114 for further recovery and processing. A part of the condensate may be conveyed via 120 via pump 122 to a chiller 128 and chilled. The chilled condensate is further conveyed and sprayed into the second fluid containing portion 108 to condense the vapor comprising the C3-C6 alcohol.

In some embodiments, methods of the present invention are directed to methods for recovery of C3-C6 alcohols from solutions such as fermentation broths in which a gas is introduced into a fermentation broth in order to effect transfer of the C3-C6 alcohol into the gas, and subsequently recovering C3-C6 alcohol from gas. For example, in one embodiment, the invention provides a method to recover a C3-C6 alcohol from a fermentation medium containing microorganisms and the C3-C6 alcohol, comprising introducing a gas into the fermentation medium such that a portion of the C3-C6 alcohol transfers into the gas; conducting the gas from the fermentation medium to a recovery unit; and recovering the C3-C6 alcohol from the gas. In this embodiment, the gas can be any suitable gas for recovering the C3-C6 alcohol, including air, carbon dioxide, or nitrogen.

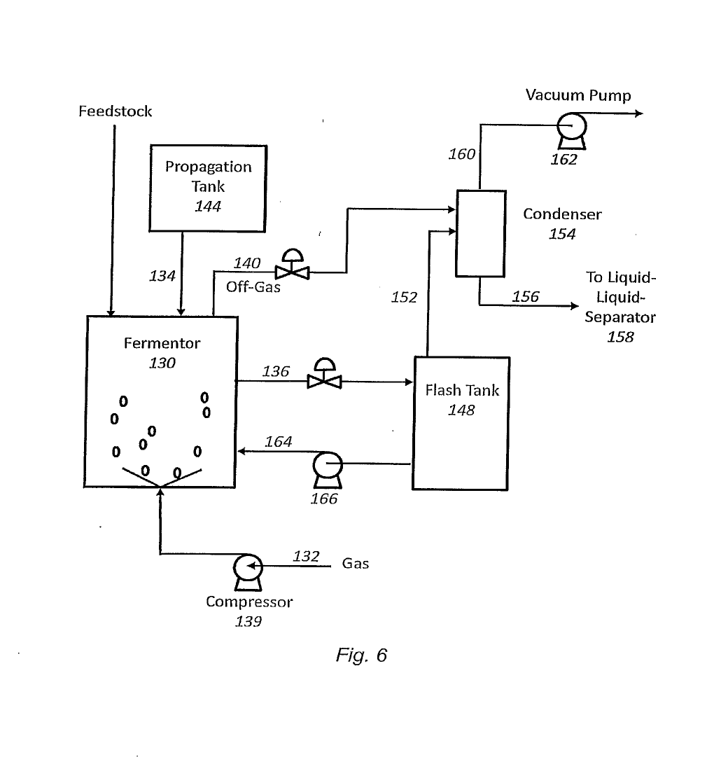

With reference to FIG. 6, an embodiment of the present invention including a means for applying gas stripping (or scalping) to recover C3-C6 alcohols from a fermentation broth is illustrated. Gas stripping can enhance the recovery of C3-C6 alcohol when used in conjunction with flash recovery. Fermentation is conducted in fermentor 130. The fermentation broth in the fermentor 130 includes the C3-C6 alcohol product, and other components of the fermentation medium. A propagation tank 144 conducts an initial culture to the fermentor 130 via 134. Gas stripping may take place in the fermentor 130 or in the flash tank 148. Accordingly, as shown in FIG. 6, in some embodiments, a gas is sparged via 132 and a compressor 139 in a fermentor 130 through the fermentation broth comprising microorganisms and the C3-C6 alcohol. In some embodiments the gas may be air. In some embodiments the gas may be a nonreactive gas that does not react with the C3-C6 alcohol, such as nitrogen or carbon dioxide. The C3-C6 alcohol in the fermentation broth diffuses into the sparged gas bubbles and exits the fermentor as part of the exhaust gas via 140 and is conveyed via 140 to a vapor condenser 154.

During the course of the fermentation, a stream of the fermentation broth, which may include microorganisms, is conducted from the fermentor 130 to the flash tank 148. The C3-C6 alcohol comprised in the flash tank vapor is combined with the sparged gas bubbles in the condenser 154 to join the flash vapor traffic. The C3-C6 alcohol can then be recovered from the flash vapor. The portion of the vaporized fermentation broth includes only a portion of the alcohol in the fermentation broth along with water vapor and sparged gas. The portion of the fermentation broth that is vaporized in the flash tank 148 is conducted as a vapor to a vapor condenser 154 via 152. Upon condensation of the mixed alcohol and vapor, the condensed solution is conducted to a liquid-liquid separator 158 via 156. The remaining vapor that is not condensed is then further conducted to an outlet via 160 and pump 162. After distillation in the flash tank 148, the remaining portion of the fermentation broth that is not distilled is returned to the fermentor 130 via 164 and pump 166. This fermentation broth that is being returned to the fermentor is now partially depleted of alcohol.

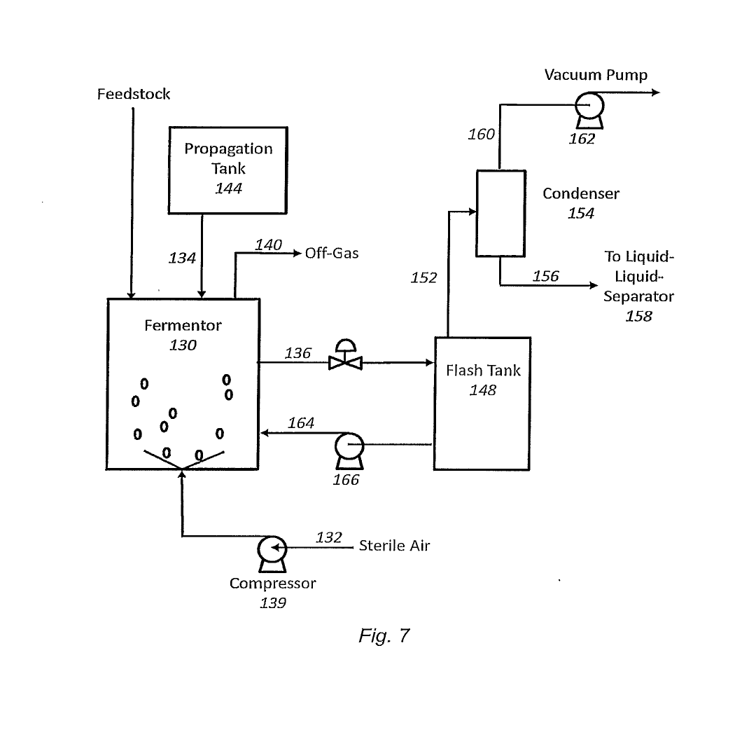

FIG. 7 illustrates an embodiment of this invention, in which sterile air comprising oxygen is introduced into the fermentor. Fermentation is conducted in fermentor 130. The fermentation broth in the fermentor 130 includes the C3-C6 alcohol product, and other components of the fermentation medium. A propagation tank 144 conducts an initial culture to the fermentor 130 via 134. Sterile air is sparged via 132 and a compressor 139 in the fermentor 130 through the fermentation broth comprising microorganisms and the C3-C6 alcohol. C3-C6 alcohol in the fermentation broth diffuses into the sparged air bubbles and exits the fermentor as part of the exhaust gas via 140. The C3-C6 alcohol can be recovered from the off gas such as by combining it with vapor from the flash tank 148 in the condenser 154 or by capturing the C3-C6 alcohol in a scrubber.

During the course of the fermentation, a stream of the fermentation broth, which may include microorganisms, is conducted from the fermentor 130 to the flash tank 148. The C3-C6 alcohol can then be recovered from the flash vapor. The portion of the vaporized fermentation broth includes only a portion of the alcohol in the fermentation broth along with water vapor. The portion of the fermentation broth that is vaporized in the flash tank 148 is conducted as a vapor to a vapor condenser 154 via 152. Upon condensation of the mixed alcohol and vapor, the condensed solution is conducted to a liquid-liquid separator 158 via 156. The remaining vapor that is not condensed is then further conducted to an outlet via 160 and pump 162. After distillation in the flash tank 148, the remaining portion of the fermentation broth that is not distilled is returned to the fermentor 130 via 164 and pump 166. This fermentation broth that is being returned to the fermentor is now partially depleted of alcohol.

Other aspects of fermentation methods described herein can be advantageously combined with this embodiment, such as any of the following, either alone or in combination:

culturing a microorganism in a fermentation medium to produce the C3-C6 alcohol; and conducting the water rich phase to the fermentation medium;

hydrolyzing a feedstock containing a polysaccharide and at least one other compound to produce fermentable hydrolysis products and subsequent steps as described elsewhere herein;

distilling a vapor phase containing water and the C3-C6 alcohol; and reacting the C3-C6 alcohol in the vapor phase to form a product;

increasing the activity of the C3-C6 alcohol in a portion of the fermentation medium;

distilling a portion of the dilute aqueous solution to a vapor phase comprising C3-C6 alcohol and water, wherein the vapor phase comprises between about 1% by weight and about 45% by weight of the first amount of C3-C6 alcohol from the portion of the dilute aqueous solution; and condensing the vapor phase.

Another embodiment of the present invention is method to operate a retrofit ethanol production plant comprising a pretreatment unit, multiple fermentation units, and a beer still to produce a C3-C6 alcohol that includes introducing a gas into a fermentation broth in order to effect transfer of the C3-C6 alcohol into the gas, and subsequently recovering C3-C6 alcohol from gas.

In these embodiments that include introducing a gas into a fermentation broth in order to effect transfer of the C3-C6 alcohol into the gas, and subsequently recovering C3-C6 alcohol from gas, at least about 50%, at least about 60%, at least about 70%, at least about 80%, at least about 85%, at least about 90%, at least about 95% of the C3-C6 alcohol can be recovered from the gas.

In some embodiments the present invention includes culturing microorganisms in a fermentation broth to grow the microorganism to high cell densities (also referred to as growth phase or propagation phase) and further culturing the microorganisms to produce a C3-C6 alcohol (referred to as production phase). As the concentration of the C3-C6 alcohol increases in the fermentation broth, the growth of the microorganisms, as well as further production of the C3-C6 alcohol may be inhibited due to the accumulation of the C3-C6 alcohol in the fermentation broth. The process of the present invention further includes removing the C3-C6 alcohol from the fermentation broth for further recovery and processing during the steps of culturing. Removal of the C3-C6 alcohol from the fermentation broth during the growth or propagation phase reduces growth inhibition of the microorganisms due to the high concentration of the C3-C6 alcohol, thus allowing the cells to grow to higher cell densities. Removal of the C3-C6 alcohol from the fermentation broth during the production phase reduces the inhibition of the C3-C6 alcohol production by the microorganisms and allows for higher batch concentrations of the alcohol to be produced.

The present invention also provides methods for producing C3-C6 alcohols from solutions such as fermentation broths in which the culturing proceeds in two phases, growth and production, where the production phase is performed under low oxygen conditions, including anaerobic conditions. Accordingly, in one embodiment, the invention provides a method for producing a C3-C6 alcohol including culturing a microorganism in a fermentation medium to grow the microorganism, culturing the microorganism in the fermentation medium to produce the C3-C6 alcohol and recovering the C3-C6 alcohol from the fermentation medium during the steps of culturing. The method can be characterized in that a gas comprising oxygen is introduced into the fermentation medium during the step of growing the microorganism at an oxygen transfer rate (OTR) of between about 5 and about 150 mmoles of oxygen per liter of fermentation medium per hour. The method can also be characterized in that a gas comprising oxygen is introduced into the fermentation medium during the step of producing the C3-C6 alcohol at an oxygen transfer rate (OTR) of less than about 20 mmoles of oxygen per liter of fermentation medium per hour. Limiting the OTR facilitates the production of alcohol by limiting the ability of the microorganism to grow. In other embodiments, a gas containing oxygen is transferred into the fermentation medium during the step of producing the C3-C6 alcohol at an OTR of less than about 10 mmoles of oxygen per liter of fermentation medium per hour or less than about 5 mmoles of oxygen per liter of fermentation medium per hour.

It has been surprisingly found that in this embodiment, at some point in the production phase, as productivity is slowing down, productivity declines can be reversed by increasing the OTR. Without being bound by theory, it is believed that this step can revive or enhance cell growth and/or production of C3-C6 alcohol. Thus, this embodiment of the present invention can also include increasing the OTR during a production phase of a fermentation, that is, at a point in time after the OTR has been reduced from the OTR during the growth phase. More particularly, this embodiment can include introducing a gas comprising oxygen into the fermentation medium during production of the C3-C6 alcohol at an OTR in excess of the OTR required for the production C3-C6 alcohol. It should be appreciated that different production microorganisms for C3-C6 alcohols will have varied OTR requirements for production of alcohol. For example, some microorganisms can produce alcohol under anaerobic conditions whereas some may require small amounts of oxygen. More particularly, the OTR can be between about 0.5 and about 5 mmoles of oxygen per liter of fermentation medium per hour, between about 0.5 and about 4 mmoles of oxygen per liter of fermentation medium per hour, between about 0.5 and about 3 mmoles of oxygen per liter of fermentation medium per hour, between about 0.5 and about 2 mmoles of oxygen per liter of fermentation medium per hour, or between about 0.5 and about 1 mmoles of oxygen per liter of fermentation medium per hour.

OTR can be utilized to determine the consumption of oxygen per unit of fermentation volume per unit time. This information is important for correct fermentor system design and operation. OTR can be controlled to establish anaerobic, micro-aerobic and fully aerobic conditions. These various regimes of OTR can be used to establish a balanced control between growth of the organism or yield of the desired metabolite such as an alcohol. The OTR achieved in a fermentation system is dependent on several variables including but not limited to fermentor design (baffles, height to width ratio, agitation systems), gas injection system, pressure, temperature, media viscosity and composition. OTR can be determined from basic process data and calculations that characterize oxygen from the gas phase to the individual cells. Once the OTR characteristics of a given fermentation system are understood, specific controls can be manipulated to control the regime of aeration. Process variables often utilized for OTR control are gas feed rate, fermentor pressure and mixing intensity. In addition the injection gas utilized can be selected to include air or it can be a mixture of one or more purified gases. Examples of purified gases include oxygen, nitrogen and carbon dioxide. Several approaches to measure and characterize the OTR for a fermentation system have been developed. Some of the measurement methods determine the OTR without active cultures in the fermentor. Other approaches measure the OTR of the system with active culture systems. The OTR approach utilized for this body of work is the Oxygen Balance Technique in active fermentations. The oxygen consumption is determined by measuring the rate of oxygen (mMol O2/hour) supplied to the fermentor and subtracting the rate of oxygen (mMol O2/hr) exiting the fermentor. This transfer rate of oxygen is divided by the fermentation volume in liters to establish the OTR (mMol O2/L-hr). The oxygen flow rates and composition of the inlet and exit gas streams can be measured by various approaches. One established method for the measurement of gas flow rates and composition include the use of a gas flow meter and a mass spectrometer. Gas flow rates into and exiting the system are typically measured in volumetric rates per unit time and converted to molar flow rate per unit time (mMol/hr) using the ideal gas law. The mass spectrometer measures the composition of the feed and exit gases and can be used to calculate the oxygen molar flow rate (mMol O2/hr) from the total gas flow rate (mMol/hr). The fermentor volume is measured by one of many means including differential pressure level transmitters, calibrated volume sight glass and radar level gauge or other means.

In this embodiment of a method for producing a C3-C6 alcohol, the step of recovering can include increasing the activity of the C3-C6 alcohol in a portion of the fermentation medium to at least that of saturation of the C3-C6 alcohol in the portion, or decreasing the activity of water in a portion of the fermentation medium to at least that of saturation of the C3-C6 alcohol in the portion; forming a C3-C6 alcohol-rich liquid phase and a water-rich liquid phase from the portion of the fermentation medium; and separating the C3-C6 alcohol-rich phase from the water-rich phase. This embodiment can also include conducting the water rich phase to the fermentation medium. In these embodiments, increasing the activity of the C3-C6 alcohol in a portion of the fermentation medium to at least that of saturation of the C3-C6 alcohol in the portion can include distilling a vapor phase comprising water and C3-C6 alcohol from the fermentation medium and reacting the C3-C6 alcohol in the vapor phase to form a product.

The present invention includes other embodiments characterized in that a gas comprising oxygen is introduced into the fermentation medium during the step of producing the C3-C6 alcohol at an oxygen transfer rate (OTR) of less than about 20 mmoles of oxygen per liter of fermentation medium per hour. Particularly, the present invention includes a method to produce a C3-C6 alcohol that includes culturing a microorganism in a fermentation medium to produce the C3-C6 alcohol, introducing a gas comprising oxygen into the fermentation medium during the culturing step at an OTR of less than about 20 mmoles of oxygen per liter of fermentation medium per hour, increasing the activity of the C3-C6 alcohol in a portion of the fermentation medium, distilling the portion of the fermentation medium to produce a vapor phase comprising water and C3-C6 alcohol and a liquid phase, and conducting the liquid phase to the fermentation medium. A further such method is a method to operate a retrofit ethanol production plant comprising a pretreatment unit, multiple fermentation units, and a beer still to produce a C3-C6 alcohol. This method includes pretreating a feedstock to form fermentable sugars in the pretreatment unit and culturing a microorganism in a fermentation medium comprising the fermentable sugars in a first fermentation unit to grow the microorganism. The method further includes culturing the microorganism in the fermentation medium comprising the fermentable sugars in a first fermentation unit to produce the C3-C6 alcohol, while introducing a gas comprising oxygen into the fermentation medium at an OTR of less than about 20 mmoles of oxygen per liter of fermentation medium per hour. The C3-C6 alcohol is recovered by treating a portion of the fermentation medium comprising the C3-C6 alcohol to remove a portion of the C3-C6 alcohol and returning the treated portion of the fermentation medium to the fermentation unit. The method also includes transferring the fermentation medium from the fermentation unit to the beer still.

In any of these embodiments, the step of producing the C3-C6 alcohol can be anaerobic. A fermentor can be made anaerobic by stopping the introduction of air or any other oxygen-containing gas so that after any residual oxygen in the medium is used by the microorganisms, the medium will be anaerobic. Alternatively, a fermentation medium can be flushed with nitrogen, carbon dioxide or other inert gas to produce an anaerobic medium.

Other embodiments of the present invention include methods for producing and recovering C3-C6 alcohols in an energy efficient manner. In some embodiments, the present invention includes the use of eductors for heat integration, which results in reduced overall plant energy consumption and provides substantial cost savings. The eductors used in these processes are steam powered venturi devices that are used to generate vacuum. Steam under high pressure is passed through an eductor to generate a vacuum at one operation and may be used to drive other operations. Accordingly, in one embodiment the present invention includes a method for operating a process for production and recovery of a C3-C6 alcohol comprising multiple unit operations that are operated at less than atmospheric pressure. The method includes introducing steam into a first eductor to create less than atmospheric pressure at a first unit operation; and conducting steam from the first eductor to a second eductor to create less than atmospheric pressure at a second unit operation. The first and second unit operations can be the same or can be different. In a related embodiment, the invention provides a method for operating a process for production and recovery of a C3-C6 alcohol comprising multiple unit operations that are operated at successively lower pressures. The method includes the steps of introducing steam under pressure P1 into a first eductor to create less than atmospheric pressure at a first unit operation; and conducting steam and other gases (e.g., vaporized butanol and carbon dioxide) from the first eductor at a pressure P2, where P2>P1 to a second eductor to create a greater vacuum. The multiple unit operations may include any unit operation used in a process for production and recovery of a C3-C6 alcohol, including but not limited to a water reclamation, a first effect evaporator, a second effect evaporator, a beer still, side stripper and/or a rectifier.

In another embodiment, as shown in FIG. 5, steam under high pressure is passed through an eductor 136 via 133 generating vacuum in the flash tank-direct contact condenser unit 100. The heat contained in the excess steam, steam condensate and non-condensed product vapor from the flash tank-direct contact condenser unit is routed through the eductor to a beer still or evaporators 138. The heat is integrated in the production and recovery process by transfer to subsequent process steps in the beer still or in the evaporators.

The present invention also provides methods for recovery of C3-C6 alcohols from solutions such as fermentation broths in which a high cell density culturing method is employed. For example, in one embodiment, the invention provides a method to culture C3-C6 alcohol producing microorganisms to high cell densities that includes growing the microorganisms in a fermentation medium and recovering the C3-C6 alcohol from the fermentation medium during the step of growing. In this method, the microorganisms reach a cell density ranging from about 5 g per liter to about 150 g per liter dry weight. In alternate embodiments, the microorganisms may reach cell densities that vary over a range of about 5 g/l dry weight to about 150 g/l dry weight. In particular, the lower end of the range may be selected from about 5 g/l dry weight, about 15 g/l, about 25 g/l, about 50 g/l, about 75 g/l and about 100 g/l dry weight of the microorganisms and the upper end of the range may be selected from about 150 g/l, about 125 g/l, about 100 g/l, about 75 g/l, about 50 g/l and about 25 g/l dry weight of the microorganisms. These embodiments may include any one of the lower limits and any one of the upper limits.

In another embodiment, the invention provides a method to produce a C3-C6 alcohol that includes the steps of culturing microorganisms that produce the C3-C6 alcohol in a fermentation medium to produce the C3-C6 alcohol and recovering the C3-C6 alcohol from the fermentation medium; wherein the production of the C3-C6 alcohol is at a rate of at least about 1 g per liter per hour. In alternate embodiments, the production of the C3-C6 alcohol is at a rate of at least about 2 g per liter per hour. In preferred embodiments, the C3-C6 alcohol can be butanol or specifically, isobutanol.

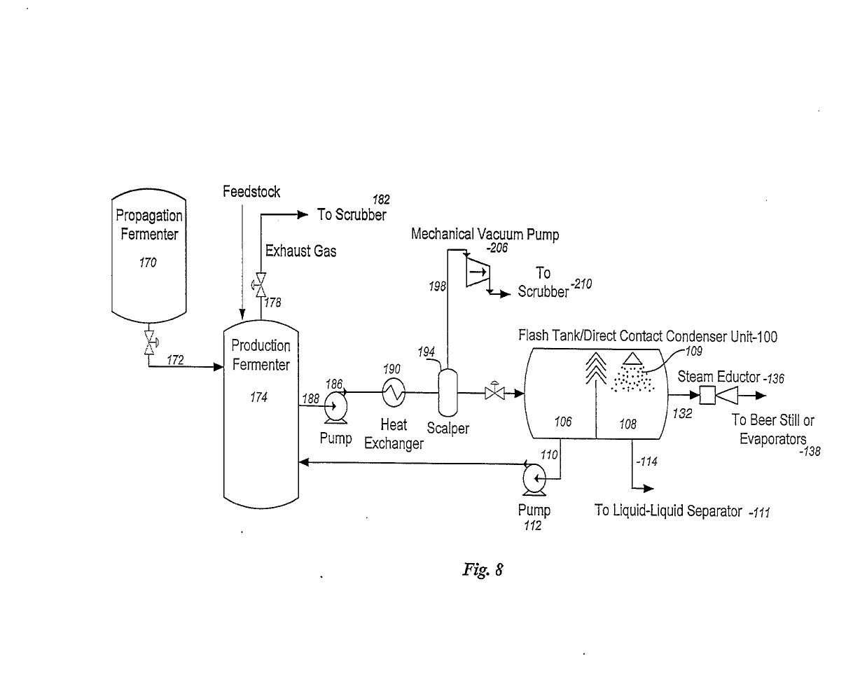

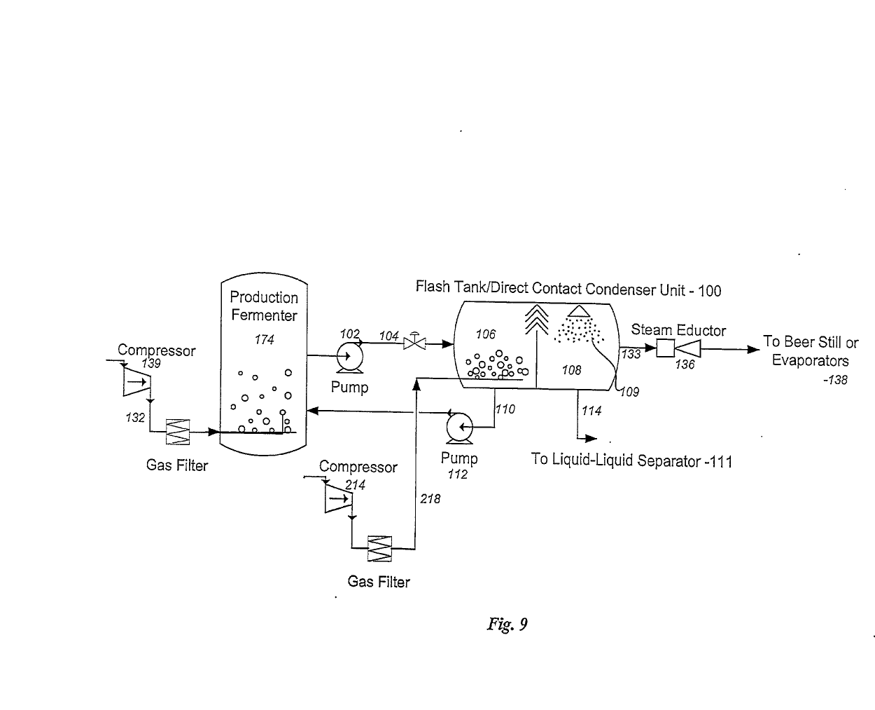

The various embodiments discussed above can be combined with each other. For example, as shown in FIGS. 8 and 9, gas scalping or gas stripping may be performed in combination with a flash tank-direct contact condenser unit to provide greater efficiencies in the alcohol recovery. FIG. 8 represents an embodiment of the present invention for the production and recovery of a C3-C6 alcohol from a fermentation broth using a flash tank/direct contact condenser unit 100 and a gas scalper. A propagation fermentor 170 conducts an initial culture to the production fermentor 174 via 172. Exhaust gas exits the fermentor to a scrubber 182 via 178. During the course of the fermentation, a stream of the fermentation broth, which may include microorganisms, is conducted from the fermentor 174 to a heat exchanger 190 then to a scalper 194 via 188. Removal of gases from the scalper is effected by a mechanical vacuum pump 206 via 198 to a scrubber 210.

A stream of the fermentation broth, which may include microorganisms, is conducted to a system 100 via 188. More specifically, the broth is further conducted to a flash tank portion 106 for distillation. The vapors produced in the flash tank portion of the system 106 are conveyed to the direct contact condenser portion of the system 108 and exposed to a fine spray of condensing liquid 109 that can contain the alcohol product to increase the condensation rate. Steam from the direct contact condenser portion of the system 108 under high pressure is passed through an eductor 136 via 132 generating vacuum in the flash tank-direct contact condenser unit 100. The heat contained in the excess steam, steam condensate and non-condensed product vapor from the flash tank-direct contact condenser unit is routed through the eductor to a beer still or evaporators 138. The remainder of the condensate not used as condensing liquid 109 is sent to a liquid-liquid separator 111 via 114. After distillation in the flash tank portion 106, the remaining portion of the fermentation broth (partially depleted of alcohol) that is not distilled can be returned to the fermentor via 110 and pump 112.

FIG. 9 represents an embodiment of the present invention for the production and recovery of a C3-C6 alcohol from fermentation broth using a flash tank/direct contact condenser unit and gas stripper. Gas is sparged via 132 and a compressor 139 to a fermentor 174 through the fermentation broth comprising microorganisms and the C3-C6 alcohol. In some embodiments the gas may be air. In some embodiments the gas may be a nonreactive gas that does not react with the C3-C6 alcohol, such as nitrogen. The C3-C6 alcohol in the fermentation broth diffuses into the sparged gas bubbles.

A stream of the fermentation broth, which may include microorganisms, is conducted to a system 100 via 104 and pump 102. More specifically, the broth is further conducted to a flash tank portion 106 for distillation. Gas is sparged via 218 and a compressor 214 to flash tank portion 106. The vapors produced in the flash tank portion of the system 106 are conveyed to the direct contact condenser portion of the system 108 and exposed to a fine spray of condensing liquid 109 that can contain the alcohol product to increase the condensation rate. Steam from the direct contact condenser portion of the system 108 under high pressure is passed through an eductor 136 via 133 generating vacuum in the flash tank-direct contact condenser unit 100. The heat contained in the excess steam, steam condensate and non-condensed product vapor from the flash tank-direct contact condenser unit is routed through the eductor to a beer still or evaporators 138. The remainder of the condensate not used as condensing liquid 109 is sent to a liquid-liquid separator 111 via 114. After distillation in the flash tank portion 106, the remaining portion of the fermentation broth (partially depleted of alcohol) that is not distilled is returned to the fermentor via 110 and pump 112.

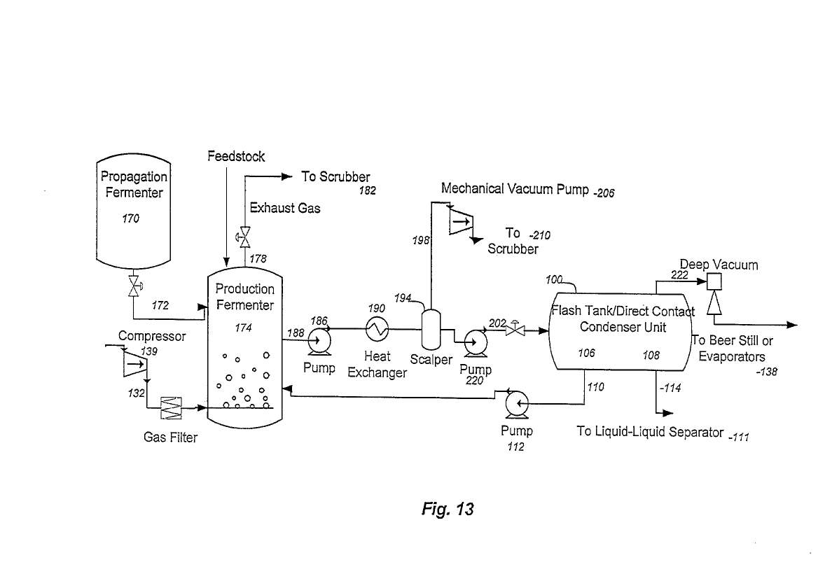

With reference to FIG. 13, a further embodiment of the present invention for the production and recovery of a C3-C6 alcohol from a fermentation broth using a flash tank/direct contact condenser unit 100 and a gas scalper and a three pump loop. A propagation fermentor 170 conducts an initial culture to the production fermentor 174 via 172. Gas is sparged via 132 and a compressor 139 to a fermentor 174 through the fermentation broth comprising microorganisms and the C3-C6 alcohol. In some embodiments the gas may be air. In some embodiments the gas may be a nonreactive gas that does not react with the C3-C6 alcohol, such as nitrogen. The C3-C6 alcohol in the fermentation broth diffuses into the sparged gas bubbles. Exhaust gas exits the fermentor to a scrubber 182 via 178.

During the course of the fermentation, a stream of the fermentation broth, which may include microorganisms, is conducted from the fermentor 174 via pump 186 to a heat exchanger 190 then to a scalper 194 via 188. Removal of gases from the scalper is effected by a mechanical vacuum pump 206 via 198 to a scrubber 210. A stream of the fermentation broth, which may include microorganisms, is conducted to a system 100 via pump 220 via 202.

A portion of the fermentation broth is vaporized in the flash tank/direct contact condenser unit 100 and the vapor is removed via 222 under vacuum to a beer still or evaporators 138. Some of the condensed vapor is sent to a liquid-liquid separator 111 via 114. After distillation in the flash tank portion 106, the remaining portion of the fermentation broth (partially depleted of alcohol) that is not distilled can be returned to the fermentor via 110 and pump 112.

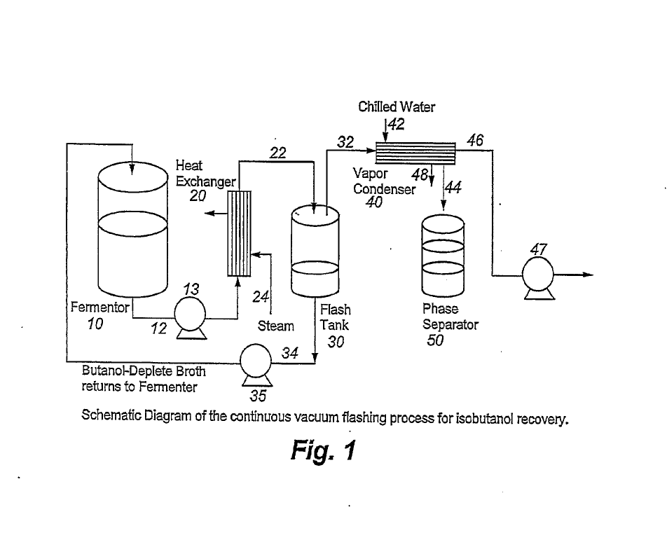

As background and context for the foregoing embodiments of the present invention, a schematic diagram of a continuous vacuum flashing process for isobutanol recovery is shown in FIG. 1. Fermentation is conducted in fermentor 10. The fermentation broth in the fermentor 10 includes the C3-C6 alcohol product, such as butanol, and other components of the fermentation medium. During the course of the fermentation, a stream of the fermentation broth, which may include microorganisms, is conducted from the fermentor 10 to a heat exchanger 20 via 12. The heat exchanger 20 is used to raise the temperature of the fermentation broth to a temperature suitable for a subsequent distillation. After the temperature of the fermentation broth is raised to an appropriate temperature, the broth is further conducted to a flash tank 30 for distillation via 22. The fermentation heat can partially supply the heat required for vaporization in the flash system. The flash tank 30 is maintained at a below atmospheric pressure so that upon introduction of the heated fermentation broth into the flash tank 30, a portion of the fermentation broth gets vaporized. The portion of the vaporized fermentation broth includes only a portion of the butanol in the fermentation broth along with water vapor. After distillation in the flash tank 30, the remaining portion of the fermentation broth that is not distilled is returned to the fermentor 10 via 34. This fermentation broth that is being returned to the fermentor is now partially depleted of butanol. The portion of the fermentation broth that is vaporized in the flash tank 30 is conducted as a vapor to a vapor condenser 40 via 32, which can be cooled, for example, by chilled water via 42. Upon condensation of the mixed butanol and water vapor, the condensed solution is conducted to a phase separator 50 via 44. The remaining vapor that is not condensed is then further conducted to an outlet via 48. The condensed solution in the phase separator is allowed to separate into a heavy liquid phase and a light liquid phase. The heavy liquid phase consists primarily of water with some amount of butanol soluble in the water. The light phase consists primarily of butanol with some amount of soluble water. From the phase separator, the light phase containing butanol can be recovered by separation from the heavy phase and can be treated for further purification. The heavy phase consisting primarily of water can be conducted for other applications or uses in the system. 13, 35 are liquid pumps and 47 is a vacuum pump.

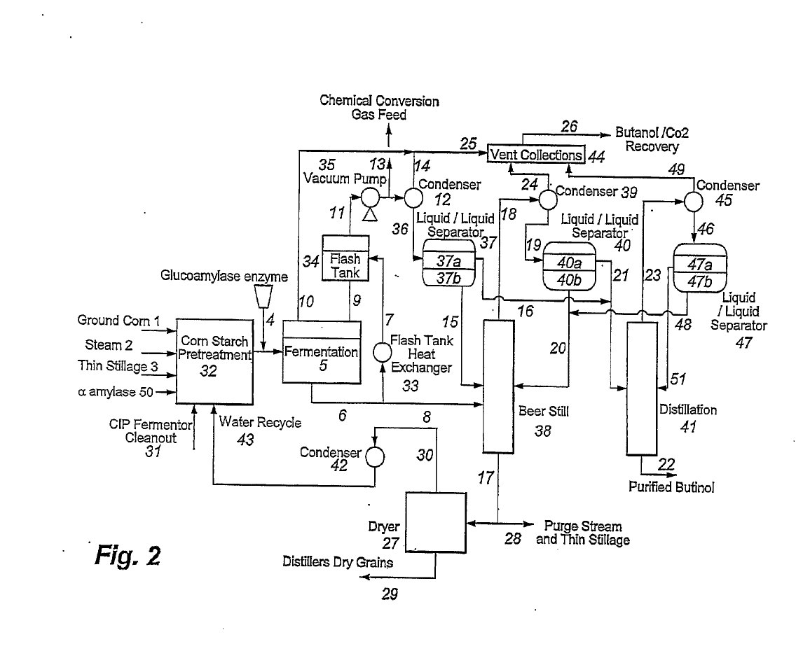

With reference to FIG. 2, and as further background and context for the foregoing embodiments of the present invention, a specific embodiment of a butanol production process by simultaneous saccharification and fermentation of pretreated corn, and azeotropic distillation of a side stream of butanol is illustrated. Dry corn is milled into a fine powder. The milled (ground) corn 1, thin stillage 3, CIP fermentor cleanout 31, recycled water 43, and steam 2 are added to a corn starch pretreatment system 32 where the mixture is slurried and heated to about 99° C. (A CIP (Clean in Place) fermentor cleanout is a caustic water solution that is used to clean and sanitize the fermentors between batches. NaOH is often used but other strong bases and other sanitization chemicals can also be used. The waste CIP solution contains solids, nutrients, carbohydrates etc from the fermentor (clinging to walls) that can be reintroduced into the front end of the corn pretreatment.) Alpha-amylase 50 is added to the corn starch pretreatment system 32 where the holding time can be about 1 hour or less. Glucoamylase enzyme 4 is added after the solution is cooled to a temperature ranging from about 50° C. to about 65° C. After a short saccharification time of about 5-6 hours the slurry is cooled to about 32° C. The slurry solids concentration at this point can be about 361 g/kg, including insoluble and soluble solids. Enzymes 4 sufficient to complete the saccharification in about 32 hours are also added to the corn mash mixture, which is transferred to the fermentor 5. The fermentation is run under simultaneous saccharification and fermentation (SSF) mode at 32° C. A side stream 6 containing about 4 wt. % butanol is continuously removed from the fermentor 5 and a flash tank heat exchanger 33 is used to control the temperature of a flash tank feed 7 at about 34° C. Vacuum of about 50 mm Hg is pulled on a flash tank 34 and an azeotropic vapor composition 11 is formed. The composition of the butanol water vapor azeotropic 11 can be about 54 wt % butanol and about 46 wt % water. The azeotrope vapor 11 is pumped by the vacuum pump 35 and is either fed to a chemical conversion process 13 or to a condenser 12. The condensed vapor phase 36 is conducted to a liquid/liquid separator 37 where it is phase separated. The condensed vapor phase separates into a butanol rich phase 37a and a water rich phase 37b. The butanol rich phase 37a has a butanol concentration of about 680 g/L butanol. The water rich phase 37b has a butanol concentration of about 86 g/L. The ratio of the volumes produced for the upper layer 37a to the lower layer 37b is 3 to 1.

The unvaporized components 9 in the flash tank 34 including cells, water, nutrients, carbohydrates, and about 2 wt % unvaporized butanol are returned to the fermentor 5. The unvaporized components 9 are depleted of butanol and when returned to the fermentor 5, can continue to produce butanol to be recovered by treatment of the side stream 6 as described above.

The water rich heavy phase 37b from the liquid/liquid separator 37 is conducted 15 to a beer still 38 and distilled. A butanol-water azeotropic composition 18 is generated in the beer still 38 and is conducted to a condenser 39 to be condensed. The condensed vapor 19 is conducted to a liquid/liquid separator 40 to be separated into a water rich heavy phase 40b and a butanol rich light phase 40a. The water rich heavy phase 40b contains about 86 g/L butanol is recycled 20 back to the beer still 38. The butanol rich phase 40a has a butanol concentration of about 680 g/L butanol.

The butanol rich light phase 40a in the liquid/liquid separator 40 is conducted 21 to a distillation system 41. The butanol rich light phase 37a in the liquid/liquid separator 37 is also conducted 16 to the distillation system 41, and can be combined with the butanol rich light phase 40a. The distillation system 41 is operated at atmospheric pressure and purified butanol is produced as a high boiling product 22 at a concentration of about 99 wt % butanol. (In other embodiments, the distillation system can be operated at sub atmospheric, atmospheric, or super atmospheric pressures.) A butanol water azeotrope vapor 23 is produced and sent to the condenser 45 and condensed. The condensed vapor 46 is conducted to a liquid/liquid separator 47 to be separated into a water rich heavy phase 47b and a butanol rich light phase 47a. The water rich heavy phase 47b is recycled 48 to the beer still 38. The butanol rich light phase 47a is conducted 51 to the distillation system 41 and can be combined with other inputs 16, 21.

The SSF fermentation in the fermentor 5 is conducted for 52 hours. The fermentation broth containing about 2% butanol that is not removed by the vacuum flash tank 34 is conducted 8 to the beer still 38. The butanol in the broth is distilled overhead as a butanol-water azeotrope 18. From the beer still 38, water, unconverted carbohydrates, nutrients, cells, fiber, corn germ, enzymes, and other fermentation components are taken as a bottoms product 17 and contains about 0.05 wt % butanol. The beer still bottoms stream 17 is divided to a distillers thy grain dryer 27 and a purge stream 28. Thin stillage 3 is produced by the purge stream 28. Dried distillers grains 29 are produced by the dryer 27. The dryer 27 also produces water vapor 30 that is condensed by a condenser 42 and recycled 43 to the corn starch pretreatment system 32.

The fermentor 5, condenser 12 (having an inflow from the flash tank 34), condenser 39 (having an inflow from the beer still 38), and condenser 45 (having an inflow from the distillation system 41) have vent streams 10, 25, 24, 49 that contain butanol, water, CO₂, and other inert gases. These streams are combined in a vent collection system 44 and are processed in downstream equipment 26 to recover and purify butanol and CO₂.

The foregoing embodiment of the invention can be conducted in a retrofit corn ethanol production plant in which the primary operations, including corn starch pretreatment system, fermentor, beer still, distillation system, and dryer are operations that previously were used to produce ethanol. Such systems have multiple fermentors (typically from five to seven) that are operated in cycle so that each one conducts a fermentation for about 52 hours before being emptied into a beer still. The operations upstream of the fermentors (e.g., the corn starch pretreatment system) operate essentially continuously preparing a feedstock for a first fermentor and then preparing a feedstock for a second fermentor and so forth. The operations downstream of the fermentors (e.g., the beer still, distillation system, and dryer) operate essentially continuously taking the fermentation broth from each fermentor as it finishes a fermentation cycle to recover ethanol, produce DDGS, a purge stream and thin stillage.

Such an ethanol production plant can be retrofit to produce butanol by incorporating various production and recovery processes described herein.

Typically, microorganisms that produce ethanol are tolerant to high concentrations of ethanol in the fermentation broth. However, high concentrations of C3-C6 alcohols in the fermentation broth can be toxic to microorganisms. Therefore, a low cost method to simultaneously remove alcohols as they are produced is required to operate an ethanol plant to produce a C3-C6 alcohol instead of ethanol.

Since butanol concentrations cannot be generated that are as high as ethanol concentrations before butanol production organisms shut down, the production and recovery processes described herein are useful for incorporation into an ethanol plant to allow efficient production of butanol. By incorporating butanol recovery processes in which a portion of a fermentation broth that can include microorganisms is taken to a recovery operation such as a flash tank for recovery of a portion of the butanol from the portion of the fermentation broth and returning a butanol-depleted stream to a fermentor, the effective butanol concentration of the fermentation can be significantly increased so that a butanol production process can be conducted into an ethanol production plant.

The process of retrofitting a plant can include introducing equipment to produce a side stream 6, flash tank feed 7, and unvaporized components stream 9, as described above into a plant. In addition, equipment for conducting liquid/liquid separations such as separators 37, 40, can be introduced to provide for efficient recovery of butanol.

Accordingly, in some embodiments, the present invention provides methods to operate a retrofit ethanol production plant utilizing method steps described in related embodiments. For example, in one embodiment, the present invention includes a method to operate a retrofit ethanol production plant to produce a C3-C6 alcohol. In this embodiment, the retrofit ethanol production plant comprises a pretreatment unit, multiple fermentation units, and a beer still to produce the C3-C6 alcohol. The method includes the steps of pretreating a feedstock to form fermentable sugars in the pretreatment unit; fermenting the fermentable sugars with a microorganism that produces the C3-C6 alcohol in a fermentation medium in a first fermentation unit; treating a portion of the fermentation medium to remove the C3-C6 alcohol; returning the treated portion to the first fermentation unit; optionally removing gases from the fermentation medium to the first fermentation unit, and transferring the fermentation medium from the first fermentation unit to the beer still.

Some methods of the present invention include the step of pretreating a feedstock to form fermentable sugars in a pretreatment unit. The pretreatment unit continuously receives the feedstock for pretreatment. The term pretreatment refers to treatments such as comminution, milling, separation of the carbon source from other components such as proteins, decrystallization, gelatinization, liquefaction, saccharification, and hydrolysis catalyzed by means of chemical and/or enzymatic catalysts. For example, the feedstock may be dry corn which may be ground, mixed with water, heated and reacted with amylases in the pretreatment unit to produce a mash or slurry containing fermentable sugars that are suitable as substrate for fermentation by micrororganisms.

Some methods of the present invention further include the step of fermenting the fermentable sugars with a microorganism that produces the C3-C6 alcohol in a fermentation medium in a first fermentation unit. A fermentation unit contains fermentation medium comprising microorganisms that are capable of converting the fermentable sugars into the C3-C6 alcohol when cultured. Such microorganisms have been described in detail above. The retrofit plant comprises multiple fermentation units. A stream of the pretreated feedstock containing fermentable sugars from the pretreatment unit is introduced into the first fermentation unit, where it is combined with the fermentation medium comprising microorganisms. The microorganisms ferment the fermentable sugars present to produce the C3-C6 alcohol.

Some methods of the present invention can further include the step of treating a portion of the fermentation medium to remove the C3-C6 alcohol. The fermentation medium comprises the C3-C6 alcohol, water, as well as the microorganisms. A portion (e.g., a side stream) of the fermentation medium from the first fermentation unit is taken to remove the C3-C6 alcohol contained therein. Treating can include any one or more of the methods for purification and recovery of C3-C6 alcohols from dilute aqueous solutions described herein and specifically, can include the steps of distilling a vapor phase comprising water and C3-C6 alcohol, addition of a hydrophilic solute, addition of a water soluble carbon source, reverse osmosis, and dialysis, and mixtures thereof, all of which steps have been described in detail above. In a preferred embodiment, this step comprises directing a sidestream from the first fermentation unit to a flash tank where the step of distilling is conducted at below atmospheric pressures. The design of a flash tank has been described in detail above.

Some methods of the present invention further include the step of returning the treated portion to the first fermentation unit. The treated portion is depleted in the C3-C6 alcohol and comprises water and can include microorganisms, both of which are returned to the fermentation medium. By removing a portion of the C3-C6 alcohol from fermentation medium and returning the medium to the fermentor, the concentration of the C3-C6 alcohol in the fermentation broth is maintained below a concentration that is detrimental to further production of the C3-C6 alcohol.

Some methods of the present invention further include the step of transferring the fermentation medium from the fermentation unit to a beer still. This step is conducted when it is desired to have the fermentation completed. Fermentation completion occurs when all fermentable carbohydrates are consumed or when the rate of carbohydrate conversion is reduced such that termination of the fermentation is desired.

In some embodiments of methods of the present invention, the rate of pretreating is the same as for the plant when it produced ethanol and/or the same as for conventional ethanol plants. As used herein, reference to a rate being the “same” includes the rate being identically the same, but also being within (plus or minus) about 25% of the rate, within about 15% of the rate, within about 10% of the rate, within about 9% of the rate, within about 8% of the rate, within about 7% of the rate, within about 6% of the rate, within about 5% of the rate, within about 4% of the rate, within about 3% of the rate, within about 2% of the rate, within about 1% of the rate. Thus, if the retrofit ethanol plant had a pretreatment rate of about 115 metric tons per hour, a pretreatment rate within about 25% of that rate would include a rate from about 7.5 tons per hour to about 12.5 tons per hour. The rate of pretreating refers to the rate at which pretreated feedstock is conducted to a fermentation unit.

In some other embodiments of these methods, the cycle time for a fermentation unit is the same as for the plant when it produced ethanol and/or the same as for conventional ethanol plants. The cycle time refers to the time from introduction of an inoculum to the time of emptying the fermentor to a beer still. For example, a typical cycle lime for a fermentor is about 52 hours.

In some embodiments, the C3-C6 alcohol output of the retrofit plant is at least about 80% of the C3-C6 alcohol equivalent of the ethanol maximum output of the plant before retrofit. In other embodiments, the C3-C6 alcohol output of the retrofit plant is at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% of the C3-C6 alcohol equivalent of the ethanol maximum output of the plant before retrofit.

The maximum output of an alcohol plant is a measure of the amount of alcohol produced by that plant, and may be expressed as gallons of alcohol produced per year or other units measuring volume or weight per time period. The output of a plant depends on the size and design of the specific plant. The term “ethanol maximum output of the plant before retrofit” refers to the maximum amount of ethanol produced by a plant or for which the plant was engineered before it is retrofit to produce a C3-C6 alcohol.

As recognized above, microorganisms used for production of ethanol are tolerant to high concentrations of ethanol in the fermentation broth, but microorganisms used for production of C3-C6 alcohols are typically not tolerant to high concentrations of C3-C6 alcohols. Advantageously, using the methods of the present invention it is possible to retrofit an ethanol plant to produce a C3-C6 alcohol at output levels comparable to that of ethanol, limited only by the theoretical conversion efficiency of that particular alcohol. The theoretical conversion efficiency of glucose to ethanol, on a weight basis, is 51% or 0.51. (In practice however, some of the glucose is used by the micro-organisms for production of cell mass and metabolic products other than the alcohol, and the actual conversion efficiency is less than the theoretical maximum.) Depending on the fermentation pathway used by the micro-organism, the theoretical conversion efficiency of glucose to propanol can range from 0.33 to 0.44, that of butanol can range from 0.27 to 0.41, that of pentanol can range from 0.33 to 0.39, and that of hexanol can range from 0.28 to 0.38. The term “C3-C6 alcohol equivalent” refers to the ratio of the theoretical conversion efficiency of a particular C3-C6 alcohol to that of ethanol and is specific for the fermentation pathway used. Thus, the “iso-butanol equivalent of ethanol” (for the pathway in which one molecule of glucose is broken into one molecule of isobutanol, two molecules of ATP and two molecules of CO₂) as used herein is 0.401÷ 0.51=0.806. For example, consider an ethanol plant with an ethanol maximum output of the plant before retrofit of about 100 million gallons/year. Using the methods of the present invention, it is possible to retrofit the plant and operate it to produce butanol at a theoretical maximum output of about 80.6 million gallons per year. However, given that the density of ethanol is 0.7894 and the density of isobutanol is 0.8106, the actual theoretical maximum output of isobutanol is about 78 million gallons per year. The exact number of gallons per year can be calculated using the density information, the theoretical yields and/or the actual practical yields achieved.

In various embodiments, an ethanol plant can be retrofit and operated at an output of at least about 80% of the theoretical maximum output for any given C3-C6 alcohol, accounting for density differences. In other embodiments, the C3-C6 alcohol output of the retrofit plant could be at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% of theoretical maximum output, accounting for density differences.

Various embodiments of the present invention include steps of culturing microorganisms in a fermentation medium and recovery from fermentation broths. The terms “fermentation” or “fermentation process” or “culturing a microorganism” are defined as a process in which a biocatalyst is cultivated in a culture medium containing raw materials, such as feedstock and nutrients, wherein the biocatalyst converts raw materials, such as a feedstock, into products. Biocatalysts, and related fermentation processes, suitable for the present invention are discussed in detail in U.S. patent application Ser. No. 12/820,505, filed Jun. 22, 2010, entitled, “Yeast Organism Producing Isobutanol at a High Yield” (Unpublished); U.S. patent application Ser. No. 12/610,784, filed Nov. 2, 2009, entitled, “Engineered Microorganisms Capable of Producing Target Compounds under Anaerobic Conditions” (Published as US 2010/0143997); PCT/US09/69390, filed Dec. 23, 2009, entitled, “Engineered Yeast Microorganisms for the Production of One or More Target Compounds” (Unpublished); U.S. Patent Application 61/350,209, filed Jun. 1, 2010, entitled, “Methods and Compositions for Increasing Dihydroxyacid Dehydratase Activity and Isobutanol Production”; U.S. Patent Application 61/304,069, filed Feb. 12, 2010, entitled, “Increased Isobutanol Yield in Yeast Biocatalysts by Elimination of the Fermentation By-Product Isobutyrate”; U.S. Patent Application 61/308,568, filed Feb. 26, 2010, entitled, “Decreased Production of the By-Product Isobutyrate During Isobutanol Fermentation Through Use of Improved Alcohol Dehydrogenase”; U.S. Patent Application 61/352,133, filed Jun. 7, 2010, entitled, “Reduction of 2,3-Dihydroxy-2-Methylbutanoic Acid (DH2 MB) Production in Isobutanol Producing Yeast”; U.S. patent application Ser. No. 12/371,557, filed Feb. 13, 2009, entitled, “Engineered Microorganisms for Producing Propanol” (Published as US 2009/0246842); U.S. Patent Application 61/292,522, filed Jan. 6, 2010, entitled, “Fermentative Process for Production of Isopropanol at High Yield”; U.S. patent application Ser. No. 11/963,542, filed Dec. 21, 2007, entitled, “Butanol Production by Metabolically Engineered Yeast” (Published as US 2010/0062505); U.S. patent application Ser. No. 11/949,724, filed Dec. 3, 2007, entitled, “Engineered Microorganisms for Producing N-Butanol and Related Methods” (Published as US 2009/0155869), which are incorporated by reference in their entirety. The biocatalyst may be any microorganism capable of converting a selected feedstock to a desired C3-C6 alcohol. Further aspects of the biocatalyst are discussed below. Any feedstock that contains a fermentable carbon source is suitable for the present invention.

The terms fermentation broth and fermentation medium are synonymous. Unless explicitly noted, the term fermentation broth should be construed to include both fermentation broth containing micro-organisms as well as fermentation broth which does not contain microorganisms. Similarly, the term fermentation broth includes both fermentation broth containing gases as well as fermentation broth which does not contain gases. Gases in the fermentation medium may be produced by microorganisms in the fermentation broth, or may be introduced into the fermentation medium, as discussed in detail below. In some embodiments, the fermentation broth contains gases and at least of a portion of the gases are removed from the fermentation broth. Gas removal is discussed in detail above.

Any feedstock that contains a fermentable carbon source is suitable for embodiments of the present invention that include a step of culturing a microorganism. Examples include feedstocks containing polysaccharides, such as starch, cellulose and hemicellulose, feedstocks containing disaccharides, such as sucrose, sugarcane juice and sucrose-containing molasses, and monosaccharides, such as glucose and fructose. Suitable feedstocks include starchy crops, such as corn and wheat, sugarcane and sugar beet, molasses and lignocellulosic material. Suitable feedstocks also include algae and microalgae. Where desired, the feedstock may undergo treatments such as comminution, milling, separation of the carbon source from other components, such as proteins, decrystallization, gelatinization, liquefaction, saccharification, and hydrolysis catalyzed by means of chemical and/or enzymatic catalysts. Such treatment can be conducted prior to fermenting or simultaneously with it, e.g. as in simultaneous saccharification and fermentation.

The fermentation broth of the present invention typically has a single liquid phase, but is not necessarily homogeneous since it may contain non-fermented insoluble solids, e.g. in a suspended form. The fermentation feedstock may contain compounds of limited water solubility and optionally also of limited or no fermentability. For example, according to an embodiment of the invention, the fermentation feedstock is comminuted corn and the carbon source is starch contained in it. Possibly, the starch is gelatinized, liquefied and/or saccharified, but insoluble components whether starchy or others (e.g. non-fermented protein) may still exist in the fermentation liquid. According to another embodiment, the fermentation feedstock is a lignocellulosic material and the carbon source is hydrolyzed cellulose and/or hemicellulose. Here again, some of the feedstock components are of limited water solubility. In these and other cases, the fermentation liquid may consist of an aqueous solution of the alcohol with solids suspended in it. Yet, according to an important aspect of the invention, in all those cases, only a single liquid phase exists in the fermentation broth.

In various embodiments of the invention that include fermentation, the step of fermentation can be conducted simultaneously with other process steps such as various recovery methods disclosed herein, that include the steps of increasing the activity of a C3-C6 alcohol and also the steps of hydrolyzing feed stocks to prepare a fermentation substrate.

In this method, the step of hydrolyzing can include any method capable of breaking polymeric carbohydrates into fermentable products. Thus, the step of hydrolyzing may be chemically or enzymatically catalyzed hydrolysis or autohydrolysis, and saccharification. In this method, the steps of hydrolyzing and fermenting can be conducted simultaneously for at least a portion of time of the method, can be conducted simultaneously for all the time of the method, or can be conducted at distinct times.

Suitable microorganisms for use in processes of the present invention can be selected from naturally occurring microorganisms, genetically engineered microorganisms and microorganisms developed by classical techniques, or a combination thereof. Such microorganisms can include, without limitation, bacteria and fungi (including yeast). For example, suitable bacteria can include those that are capable of alcohol production such as the bacteria of the Clostridium species. Examples of these include without limitation, Clostridium hutyricum, Clostridium acetobutylicum, Clostridium saccharoperbutylacetonicum, Clostridium saccharobutylicum and Clostridium beijerickii.

Suitable bacteria and fungi also include those that are capable of hydrolyzing carbohydrates and can be genetically engineered to produce alcohols. Suitable microorganisms can be selected from naturally occurring microorganisms, genetically engineered microorganisms and microorganisms developed by classical techniques, or a combination thereof. and have been discussed in detail above.

Examples include, without limitation, bacteria of the order Clostridiales (e.g. Butyrovibrio fibrisolvens), Bacilliales (e.g. Bacillus circulans), Actinomycetales (e.g. Streptomyces cellulolyticus), Fibrobacterales (e.g. Fibrobacter succinogenes), Xanthomonadales (Xanthomonas species) and Pseudomonadales (e.g. Pseudomonas mendocina) and fungi such as those of the order Rhizopus, Saccharomycopsis, Aspergillus, Schwanniomyces and Polysporus. The fungi may be able to do the conversion aerobically or anaerobically. Examples of anaerobic fungi include, without limitation, Piromyces species (e.g. strain E2), Orpinomyces species (e.g. Orpinomyces bovis), Neocallimastix species (N. frontalis), Caecomyce species, Anaeromyces species and Ruminomyces species. As noted above, any microorganism, whether naturally occurring or manmade, that is capable of producing alcohol can be used and the methods of the present invention are not limited to the examples listed here. In some embodiments, the microorganism is viable at temperatures from about 20° C. to about 95° C. Reference to a microorganism being viable at a given temperature or range of temperatures refers to a microorganism being able to survive exposure to such temperatures and subsequently be able to grow and/or produce metabolic products under the same or different conditions. In other embodiments, the microorganism is a temperature resistant microorganism. The term “resistance” is defined as the property of a biocatalyst to have a low rate of inhibition in the presence of increasing concentrations of an inhibitor in the fermentation broth. The term “more resistant” describes a biocatalyst that has a lower rate of inhibition towards an inhibitor than another biocatalyst with a higher rate of inhibition towards the same inhibitor. For example, two biocatalysts A and B, both with a tolerance of 2% to an inhibitor biofuel precursor and a specific productivity of 1 g product per g CDW per h, exhibit at 3% biofuel precursor a specific productivity of 0.5 g product per g CDW per h and 0.75 g product per g CDW per h for A and B, respectively. The biocatalyst B is more resistant than A. The term “temperature resistant” describes a biocatalyst that has a lower rate of inhibition at a given temperature than another biocatalyst with a higher rate of inhibition at the same temperature.

The term “tolerance” is defined as the ability of the biocatalyst to maintain its specific productivity at a given concentration of an inhibitor. The term “tolerant” describes a biocatalyst that maintains its specific productivity at a given concentration of an inhibitor. For example, if in the presence of 2% of an inhibitor a biocatalyst maintains the specific productivity that it had at 0 to 2%, the biocatalyst is tolerant to 2% of the inhibitor or has a tolerance to 2% of the inhibitor. The term “tolerance to temperature” is defined as the ability of the biocatalyst to maintain its specific productivity at a given temperature.

In some embodiments, the microorganism has a productivity of at least about 0.5 g/L per hour of the C3-C6 alcohol in aggregate over the lifetime of a batch fermentation cycle. In some embodiments, the productivity is at least about 1, at least about 1.5, at least about 2.0, at least about 2.5, at least about 3, at least about 3.5, at least about 4.0, at least about 4.5, and at least about 5.0 g/L per hour of the C3-C6 alcohol in aggregate over the lifetime of a batch fermentation cycle. In some embodiments, the productivity ranges from about 0.5 g/L per hour to about 5 g/L per hour of the C3-C6 alcohol over the lifetime of a batch fermentation cycle.

In other embodiments, preferred microorganisms are ones that produce the desired alcohol with no or minimal coproducts or byproducts. Also preferred are microorganisms that use simple and low cost fermentation media.

Some methods of the invention include increasing the activity of the C3-C6 alcohol in a portion of the aqueous solution to at least that of saturation of the C3-C6 alcohol in the portion. This step promotes the condition that some of the C3-C6 alcohol is no longer soluble in the aqueous solution and enables the formation of a C3-C6 alcohol-rich liquid phase and a water-rich liquid phase. Increasing the activity of the C3-C6 alcohol to at least that of saturation of the C3-C6 alcohol in an aqueous solution refers to processing a portion of the aqueous solution to form a composition comprising C3-C6 alcohol in which the effective concentration of the C3-C6 alcohol with respect to the aqueous solution is greater than in the starting portion. Such processing can encompass a variety of process steps including, but not limited to addition of a hydrophilic solute, distilling a vapor phase comprising water and the C3-C6 alcohol, reverse osmosis, dialysis, selective adsorption and solvent extraction. Such steps are explained in detail below. The activity of a C3-C6 alcohol refers to the effective concentration of the C3-C6 alcohol in an aqueous solution. The term saturation of the C3-C6 alcohol in the aqueous solution refers to the maximum concentration of the C3-C6 alcohol under the conditions (e.g. temperature and pressure) of that aqueous solution. As used herein, reference to a “portion” of a thing, such as a fermentation broth, includes both the entire thing (e.g., an entire fermentation broth) or some part of the entire thing that is less than the entire thing (e.g., a sidestream of a fermentation broth). A portion of a solution or fermentation broth also includes the solution or fermentation broth if it is converted to vapor phase. The activity of the C3-C6 alcohol will depend on temperature, pressure, and composition. The activity of a species can be changed or modified because molecules in a non-ideal solution, such as a fermentation medium interact with each other and interact differently with different types of molecules.

An example of increasing the activity of an alcohol is when an alcohol is removed selectively compared with water to form another phase, such as by distillation, extraction and adsorption where the other phase is gaseous, solvent phase and solid adsorbent phase, respectively. Upon condensation of the gaseous phase, separation from the solvent or separation from the adsorbent, a second liquid phase is formed in which the activity of the alcohol is higher than starting solution. An example of decreasing water activity is when water is removed selectively compared with alcohol to form another phase, such as selective adsorption, extraction and even freezing of water. The result is decreasing the activity of water in the starting solution. Some processes both increase the activity of an alcohol and decrease the activity of water. For example, if a hydrophilic solute is added to an aqueous solution of an alcohol, it leads to both decreasing water activity and increasing the alcohol activity.

According to an embodiment of the invention, increasing the activity of the C3-C6 alcohol may comprise adding a hydrophilic solute to the aqueous solution. In some embodiments, the hydrophilic solute may be a water soluble carbon source. For example, if a hydrophilic solute is introduced into an aqueous isobutanol solution, the hydrophilic solute will interact with greater affinity with the water in the solution than with the isobutanol. The activity of the isobutanol in the solution will thereby be increased. The activity coefficient for a compound in an aqueous solution is an indicator of what concentration of that compound will be in a vapor phase in equilibrium with the solution and is a function of the concentration of the compound in water. The activity of a compound in a solution is the product of the concentration of the compound and its activity coefficient. For example, in an isobutanol-water mixture, the activity coefficient for isobutanol is higher than water. Therefore, the concentration of isobutanol in the vapor phase in equilibrium with the aqueous solution will be higher than in the solution.

In some embodiments in which the aqueous solution is a fermentation broth, the hydrophilic solute may be added to the entire fermentation broth in the fermentor or to a partial stream taken from the fermentor, either with microorganisms in the broth or after removal of them. Reference to adding a hydrophilic solute can refer to increasing the concentration of a hydrophilic solute already existing in the portion of the solution or to addition of a hydrophilic solute that was not previously in the solution. Such increase in concentration may be done by external addition. Alternatively, or additionally, increasing concentration may also be conducted by in situ treatment of the solution, such as by hydrolyzing a solute already existing in the solution, e.g. hydrolyzing proteins to add amino acids to the solution, hydrolyzing starch or cellulose to add glucose to the solution and/or hydrolyzing hemicellulose to add pentoses to the solution. According to another preferred embodiment, the hydrophilic solute may be one that has a nutritional value and optionally ends up in a fermentation coproduct stream, such as distillers dried grains and solubles (DDGS). In addition or alternatively, the hydrophilic solute can be fermentable and can be transferred with the water-rich liquid phase to the fermentor.

Sufficient hydrophilic solute is added to enable the formation of a second liquid phase, either solely by addition of the hydrophilic solute or in combination with other process steps. The required amount depends on the chemical nature of the alcohol, typically decreasing with increasing number of carbon atoms in the alcohol and being smaller for normal alcohols and linear ones compared with secondary or tertiary alcohols and branched ones. The required amount further decreases with increasing concentration of the alcohol in the fermentation liquid and possibly also with increasing concentration of other solutes there. The amount required in each case can be determined, in view of the present invention, experimentally.

Preferred hydrophilic solutes are those that have a strong effect of lowering the water partial vapor pressure of aqueous solutions. The added hydrophilic solute may be a salt, an amino acid, a water-soluble solvent, a sugar or combinations of those.

Preferred water soluble carbon source are those that have a strong effect of lowering the water partial vapor pressure of aqueous solutions and ones that are well fermented. The added water soluble carbon source may be a carbohydrate such as a monosaccharide, a disaccharide or an oligosaccharide and their combinations. Such saccharide may comprises hexoses, e.g. glucose and fructose and pentoses (e.g. xylose or arabinose) and their combination. Also suitable is a precursor of such carbohydrate, such as starch, cellulose, hemicellulose and sucrose or combinations of those.

In related embodiments, the hydrophilic solute can be recovered. For example, if the dilute aqueous solution is fermentation broth and the hydrophilic solute added to increase the activity of the C3-C6 alcohol in the fermentation broth is CaCl₂, then CaCl₂, after formation of alcohol-rich and water-rich liquid phases, will be primarily found the water-rich liquid phase and can be recovered from therefrom. As another example, if the dilute aqueous solution is a portion of a fermentation broth and a water soluble carbon source added to increase the activity of the C3-C6 alcohol in the fermentation broth is glucose, then glucose will be primarily found in a water-rich liquid phase and can be conducted back to the fermentation broth to provide carbon for fermentation.

In some embodiments, the method includes distillation such that the C3-C6 alcohol and water are vaporized to form an alcohol-depleted liquid phase and an alcohol-enriched vapor phase. The step of distillation can be accomplished by increasing the temperature of the aqueous solution, reducing the atmospheric pressure on the aqueous solution or some combination thereof. In some embodiments, in which the portion of the aqueous solution is a portion of a fermentation broth, the step of distilling can be conducted in a fermentation vessel.

In these embodiments, the C3-C6 alcohol concentration in the vapor phase is greater than in the aqueous solution. According to a preferred embodiment, C3-C6 alcohol concentration in the vapor phase is at least about 5 times greater than the concentration in the aqueous solution, preferably about 10 times, preferably about 15 times, preferably about 20 times, preferably about 25 times, and preferably about 30 times. The vapor phase may be condensed, such as at conditions selected so that immiscible alcohol-rich and water-rich (i.e., alcohol-poor) solutions are formed.

Distilling can be conducted at below atmospheric pressure, at about atmospheric pressure or above atmospheric pressure. Reference herein to atmospheric pressure is to atmospheric pressure at sea level and unless otherwise specified, all pressures expressed herein are absolute pressures. Suitable below atmospheric pressures include pressures from about 0.025 bar to about 1.01 bar, from about 0.075 bar to about 1.01 bar, and from about 15 bar to about 1.01 bar. Suitable above atmospheric pressures include pressures from about 1.01 bar to about 10 bar, from about 1.01 bar to about 6 bar, and from about 1.01 bar to about 3 bar.

In the embodiment when the distilling is conducted at below atmospheric pressures, the temperature can be between about 20° C. and about 95° C., between about 25° C. and about 95° C., between about 30° C. and about 95° C., or between about 35° C. and about 95° C.

In a further embodiment, in which the aqueous solution is a portion of a fermentation broth and comprises microorganisms, and in which the step of distilling is conducted in a distillation vessel, the portion of the fermentation broth is at the temperature of between about 20° C. and about 95° C., between about 25° C. and about 95° C., between about 30° C. C and about 95° C., or between about 35° C. and about 95° C. prior to introduction into the distillation vessel. In another embodiment, the temperature of the portion of the fermentation broth is brought to the desired value after it is introduced in the distillation vessel. Preferably, microorganisms are used that are viable, and even more preferably, both viable and productive at these temperatures.

Optionally, after the step of distilling, the alcohol-depleted remaining portion of the fermentation broth can be conducted from the distillation vessel to a fermentation vessel. Optionally, the alcohol-depleted remaining portion of the fermentation broth can be mixed with water, with feedstock and/or possibly other nutrients to form the culture medium for further fermentation.

In the case where the step of increasing the activity of the C3-C6 alcohol comprises distilling a vapor phase comprising water and the C3-C6 alcohol and condensing the vapor phase, the method can also include treating the portion of the dilute aqueous solution for decreasing water activity. In various embodiments, decreasing water activity comprises water removal before the step of distilling or simultaneously with the step of distilling. The step of treating can include selective removal of water, selective binding of water or selective rejection of water. According to various embodiments, the step of treating can include addition of a hydrophilic solute, addition of a carbon source, reverse osmosis, dialysis, adsorption of the alcohol on a selective adsorbent, extraction of the alcohol into a selective extractant, adsorption of water on a selective adsorbent, or extraction of water into a selective extractant.

In a preferred embodiment, the step of distilling is conducted in a flash tank, that can be operatively connected to a fermentation vessel and the process can further comprise circulating the culture medium from the fermentation vessel to the flash tank, and circulating the culture medium from the flash tank to the fermentation vessel. A flash is a one stage distillation where the vapor and liquid outlet from the flash system are in equilibrium with each other and the temperature and pressure of each phase is nearly identical. Distillation, on the other hand, comprises a series of flash stages strung together sequentially. During distillation i.e. a multi stage flash system, such as a distillation column, the vapor that comes out the top and the liquid that comes out the bottom leave at different temperatures than in a flash.

According to another embodiment, the process includes reducing pressure in a distillation vessel compared with that in the fermentation vessel. Such a pressure reduction coupled with adiabatic vaporization allows for removal of heat from the portion of the fermentation broth of the aqueous solution generated in the fermentation vessel within the distillation vessel. Alternatively or in addition, the process can include increasing pressure on the aqueous solution from the distillation vessel in the fermentation vessel. Such a pressure increase creates heat, which can be used to preheat the system at various points. For example, the heat can be used to preheat the feed in the flash tank, the beer still and/or the distillation column and can also be used in the evaporators used to concentrate the thin stillage to syrup. These components are discussed in detail below.

In a preferred embodiment, when the step of increasing the activity of the C3-C6 alcohol comprises distilling a vapor phase comprising water and the C3-C6 alcohol, the mixed vapor includes an azeotropic composition. Azeotropes are formed when molecular forces cause two or more molecular species to behave as a new vapor or/liquid species. Azeotropes are generally viewed as a limitation by chemical process industries because the azeotrope composition “pinch point” prevents the distillation of the mixture into pure components. Instead of producing pure components from the distillation process, the azeotrope manifests itself as an azeotropic composition at the top of the distillation column, as a minimum boiling point azeotrope, or from the bottom of the distillation column, as a maximum boiling point azeotrope.

When fermentation products form a maximum boiling point azeotrope with water, all of the non-azeotrope bound water must be vaporized and distilled overhead. Products within fermentation broth are typically dilute. As a result, when maximum boiling point azeotropes are formed, the amount of energy required to boil up and remove the excess un-bound water is a large heat load and can often make the vaporization and condensation processes of distillation uneconomical. Additionally, the maximum boiling point azeotrope occurs at temperatures above the boiling points of the pure species, elevating the bottom temperatures in the distillation system. As a result, the bottoms product in the maximum boiling point experiences a higher heat history than the pure species. This high temperature heat history can degrade the value of the primary product and co-products of fermentation. Distiller's dry grains and solubles (DDGS), which are typically used as a feed ingredient, are one example of such a co-product which can be degraded with exposure to high heat and lose nutritional values.

Minimum boiling point azeotropes are also known as positive azeotropes because the azeotrope has an activity coefficient of greater than 1. Maximum boiling point azeotropes are also referred to as negative azeotropes because their activity coefficient is less than 1 The magnitude of the activity coefficient dictates the degree of non-ideal activity of the azeotropic entity. This non-ideality and difficulty in separation of azeotropes has been studied. The activity coefficient is not fixed but is a function of concentration of the compound in water. As a result, the solution boiling point of the azeotrope composition varies as the concentration of the component varies. As a result, the increased pressure drop in multistage distillation columns result in higher temperature profiles at the same overhead vacuum level.

According to a preferred embodiment, an aqueous solution of the C3-C6 alcohol forms a minimum boiling point azeotrope. According to a related preferred embodiment, the concentration of the C3-C6 alcohol in the mixed vapor is substantially equal to the concentration of the alcohol in the minimum boiling point azeotrope at the pressure selected for distillation. In some particularly preferred embodiments, the concentration of the C3-C6 alcohol in the mixed vapor is greater than the concentration of the alcohol in the minimum boiling point azeotrope, as in some cases where the aqueous solution comprises other solutes in addition to the alcohol that affect the water partial vapor pressure.

Some azeotropes are known to be stable under a broad range of operating pressures, while other azeotrope systems can be “broken” by low and high pressure. For example, the ethanol-water azeotrope is broken at pressures less than 70 torr. For azeotropes that can be broken under vacuum, the use of distillation columns is sometimes limited due to the fact that the vacuum distillation columns require that the pressure drop in the distillation column is significant enough that it requires deeper vacuum to be pulled at the vacuum source. For example, attempting to maintain the vacuum distillation column feed pressure to 150 mm Hg requires that the pressure drop in the column be very small so as to ensure that the vacuum pump can maintain proper vacuum levels. To achieve low pressure drop in vacuum columns with multiple trays requires small liquid heights on the distillation trays. The low pressure drop and low liquid height in the column typically increases the column capital cost by increasing the diameter of the column.

In some embodiments, the step of increasing die activity of the C3-C6 alcohol comprises dialysis. Dialysis works on the principle of diffusion of solutes and ultra-filtration of fluid across a semi-permeable membrane. Any membrane separation system that selectively removes water from the aqueous solution is suitable for the process of the present invention. According to a preferred embodiment, dialysis is conducted in a system comprising two or more compartments. The aqueous solution of the alcohol is introduced into one and water from this solution transfers selectively through the membrane into the other. According to a preferred embodiment, the water transfer is induced by osmotic pressure. The water-receiving compartment contains a hydrophilic compound, e.g. CaCl₂ or a carbohydrate, or a concentrated solution of such compound. A concentrated solution is formed in the water-receiving compartment. That solution is treated according to various embodiments to regenerate the solute or its concentrated solution, or for other applications. Regeneration can be done by known means such as water distillation. In the case where the solute is a carbohydrate or another source of fermentable carbon, the solution can be used provide fermentables to the fermentation step.

In some embodiments, the step of increasing the activity of the C3-C6 alcohol comprises reverse osmosis. In reverse osmosis, the aqueous solution is contacted in a first compartment with a reverse osmosis membrane under pressure, whereby water selectively transfers through the membrane to a second compartment, while the alcohol is retained in the first compartment. As a result of selective water transfer into the second compartment, the concentration (and activity) of the alcohol in the liquid of the first compartment increases and preferably reaches saturation, whereby a second phase is formed in that first compartment. That compartment comprises according to this embodiment two liquid phases one of which is an alcohol-saturated aqueous phase and the other is a water-saturated alcohol solution.

In some embodiments, the step of increasing the activity of the C3-C6 alcohol comprises solvent extraction. In solvent extraction, the aqueous solution is contacted with another liquid phase (solvent or extractant), wherein at least one of water and the alcohol are not fully miscible. The two phases are mixed and then allowed to settle. According to one embodiment, the step of increasing the activity of the C3-C6 alcohol comprises extraction of the C3-C6 alcohol into an alcohol-selective extractant. The term “alcohol-selective extractant” means an extractant preferring alcohol over water so that the alcohol/water ratio in the extractant is greater than in the remaining aqueous solution. Thus, the alcohol-selective extractant or solvent is selective to the alcohol (similarly or more hydrophobic than the alcohol) and the alcohol transfers preferentially into the extractant or solvent to form alcohol-containing extractant or solvent, also referred to as extract. In some preferred embodiments, the alcohol-selective solvent may be butylacetate, tributylphosphate, decanol, 2-hepanone or octane. In another embodiment, the step of increasing the activity of the C3-C6 alcohol comprises extraction of water into a water-selective extractant. The term “water-selective extractant” means an extractant preferring water over alcohol so that the alcohol/water ratio in the extractant is lower than in the remaining aqueous solution. Thus, the water-selective extractant or solvent is selective to water (more hydrophilic than the alcohol), so that water transfers preferentially into the water-selective extractant or solvent.

In a preferred embodiment the alcohol-selective solvent can be an acidic, amine-based extractant. Such an extractant can be prepared by mixing an amine with a diluent and contacting the mixture with an acid. Amines that are suitable for forming the extractant include primary, secondary, tertiary and quaternary amines, and preferably include primary, secondary, tertiary amines. Suitable amines are also water-insoluble in both free and salt form (i.e. when an acid is bound to them). Preferably the aggregate/total number of carbon atoms on the amines is at least 20. Both aliphatic and aromatic amines are suitable and aliphatic ones are preferred. The diluent can be a hydrocarbon or another non-reactive organic solvent with boiling point of at least about 60° C., and preferably at least about 80° C. The acid can be any strong acid, such as one with a pKa (−log dissociation constant) of not greater than 3, and can either be a mineral acid or an organic acid. In one example, the amine can be trioctyl amine, the acid can be sulfuric acid and the dilent can be decane. The acid is extracted (binds to the amine) to form the extractant.

In some embodiments the step of increasing the activity of the C3-C6 alcohol comprises adsorption of the C3-C6 alcohol or water on a selective adsorbent. In adsorption, the aqueous solution is contacted with a selective adsorbent that has greater selectivity for either alcohol or water. In one embodiment, the step of increasing the activity of the C3-C6 alcohol comprises adsorption of the C3-C6 alcohol on an alcohol-selective adsorbent. An “alcohol-selective adsorbent” means an adsorbent preferring alcohol over water so that the alcohol/water ratio on the adsorbent is greater than in the remaining aqueous solution. In another embodiment, the step of increasing the activity of the C3-C6 alcohol comprises adsorption of water on a water-selective adsorbent. A “water-selective adsorbent” means an adsorbent preferring water over alcohol so that the alcohol/water ratio on the adsorbent is lower than in the remaining aqueous solution. Thus, the aqueous phase is contacted with a water-selective adsorbent, a water-carrying adsorbent is formed and the aqueous solution is enriched in the C3-C6 alcohol. According to various embodiments, the water adsorbent is hydrophilic, has surface functions capable of forming hydrogen bonds and/or has pores suitable in size to the size of water molecules. In some embodiments the adsorbent may be solid. According to a preferred embodiment, a fermentation feedstock, such as ground corn may be the adsorbent. For example, the feedstock may be contacted with the aqueous solution to selectively adsorb water out of it. In some embodiments the adsorbent may be a molecular sieve.

Some methods further includes the step of forming a C3-C6 alcohol-rich liquid phase and a water-rich liquid phase from the portion of the aqueous solution which has been treated to increase the activity of the C3-C6 alcohol. As used here, the term “alcohol-rich liquid phase” means a liquid phase wherein the alcohol-to-water ratio is greater than that in the portion of the aqueous solution. The term “water-rich liquid phase” means a liquid phase wherein the water-to-alcohol ratio is greater than that of the alcohol-rich liquid phase. The water-rich phase is also referred to in the following as alcohol-lean phase. The step of forming the two phases can be active. For example, in some embodiments, the step of forming may comprise condensing a distilled vapor phase that forms two phases after condensation. Alternatively or in addition, chilling or cooling the treated portion of the aqueous solution can result in the formation of the two phases. Other steps for actively forming the two phases can include using equipment shaped to promote the separation of phases. Separation of the phases can be accomplished in various unit operations including liquid-liquid separators comprising a liquid/liquid separator utilizing specific gravity differences between the phases and a water boot, g-force separation as in a centrifuge, or centrifugal liquid-liquid separators. Also suitable are settlers as in mixer-settler units used for solvent extraction processes. In some embodiments the step of forming is passive and may simply be a natural consequence of the previous step of increasing the activity of the C3-C6 alcohol to at least that of saturation.

In the alcohol-rich liquid phase, the ratio of the concentration of the C3-C6 alcohol with respect to the water is effectively greater than in the starting portion. In the water-rich phase, the ratio of concentration of the C3-C6 alcohol with respect to water is effectively less than in the alcohol-rich liquid phase. The water-rich phase may also be referred to as the alcohol-poor phase.

In some embodiments, the C3-C6 alcohol is propanol and the weight ratio of propanol to water in the alcohol-rich phase is greater than about 0.2, greater than about 0.5, or greater than about 1. In some embodiments, the C3-C6 alcohol is butanol and the ratio of butanol to water in the alcohol-rich phase is greater than about 1, greater than about 2, or greater than about 8. In some embodiments, the C3-C6 alcohol is pentanol and the ratio of pentanol to water in the alcohol-rich phase is greater than about 4, greater than about 6, or greater than about 10.

The concentration factor or enrichment factor for a given phase can be expressed as the ratio of alcohol to water in that phase divided by the ratio of alcohol to water in the dilute aqueous solution. Thus, for example, the concentration or enrichment factor for the alcohol-rich phase may be expressed as the ratio of alcohol/water in the alcohol-rich phase divided by that ratio in the aqueous dilute solution.

In some embodiments, the ratio of the C3-C6 alcohol to water in the C3-C6 alcohol-rich phase is greater than the ratio of the C3-C6 alcohol to water in the fermentation broth by at least about 5 fold, at least about 25 fold, at least about 50 fold, at least about 100 fold, or at least about 300 fold.

The process further includes separating the C3-C6 alcohol-rich phase from the water-rich phase. Separating the two phases refers to physical separation of the two phases and can include removing, skimming, pouring out, decanting or otherwise transferring one phase from another and may be accomplished by any means known in the art for separation of liquid phases.

In some embodiments, the method further comprises the step of cooling the C3-C6 alcohol-rich phase to increase the ratio of the C3-C6 alcohol to water in the alcohol-rich phase.

In some embodiments, the method further comprises recovering the C3-C6 alcohol from the alcohol-rich phase. Recovering refers to isolating the C3-C6 alcohol from the alcohol-rich phase. Recovering also includes enriching or increasing the concentration of the C3-C6 alcohol in the alcohol-rich phase. In various embodiments, this step may comprise a process selected from the group consisting of distillation, dialysis, water adsorption (e.g., such as use of molecular sieves), solvent extraction, contact with a hydrocarbon liquid that is immiscible in water and contact with a hydrophilic compound to produce a first phase comprising the C3-C6 alcohol and water and a second phase comprising C3-C6 alcohol, wherein the ratio of water to C3-C6 alcohol in the second phase is less than in the first phase. In preferred embodiments, the second phase comprises at least about 80%, about 85%, about 90%, about 95% or about 99% by weight C3-C6 alcohol. As used herein a liquid that is immiscible in water has a miscibility in water of less than about 1 wt %.

Methods of distillation and dialysis are discussed above with respect to the step of increasing the activity of C3-C6 alcohols and similar processes can be used to recover C3-C6 alcohol from a C3-C6 alcohol-rich phase. Regarding the use of water adsorption to recover C3-C6 alcohol from a C3-C6 alcohol-rich phase, the alcohol-rich phase is contacted with an adsorbent that selectively adsorbs water out of the alcohol rich phase. A water-carrying adsorbent is formed and the alcohol-rich phase is further enriched in the C3-C6 alcohol. According to various embodiments, the water adsorbent is hydrophilic, has surface functions capable of forming hydrogen bonds and/or has pores suitable in size to the size of water molecules. In some embodiments the adsorbent may be solid. According to a preferred embodiment, a fermentation feedstock, such as ground corn may be the adsorbent. For example, the feedstock may be contacted with the C3-C6 alcohol-rich phase to selectively adsorb water out of it. In some embodiments the adsorbent may be a molecular sieve.

Solvent extraction can also be used to recover C3-C6 alcohol from a C3-C6 alcohol-rich phase. In solvent extraction, the alcohol-rich phase is contacted with another liquid phase (solvent), wherein at least one of water and the alcohol are not fully miscible. The two phases are mixed and then allowed to settle. According to one embodiment, the solvent is selective to water (more hydrophilic than the alcohol), water transfers preferentially to the solvent phase and the alcohol-to-water ratio in the other phase increases. According to another embodiment, the solvent is selective to the alcohol (similarly or more hydrophobic than the alcohol). In some preferred embodiments the alcohol-selective solvent may be butylacetate, tributylphosphate, decanol, 2-hepanone or octane. The alcohol transfers preferentially into the solvent. In a following step, the alcohol is separated from the solvent in a form having higher alcohol-to-water ratio compared with that of the alcohol-rich phase.

Contact with a hydrocarbon liquid that is immiscible in water can also be used to recover C3-C6 alcohol from a C3-C6 alcohol-rich phase. Such liquids are hydrophobic solvents and act as described above for hydrophobic solvents, i.e. extracting the alcohol from the alcohol-rich phase. Examples of such hydrocarbon liquids include gasoline, crude oil, Fischer Tropsch materials and biofuels.

Contact with a hydrophilic compound can also be used to recover C3-C6 alcohol from a C3-C6 alcohol-rich phase. This method for recovery is similar to that described above for use of a hydrophilic compound to increase alcohol activity or to decrease water activity.

In a further embodiment of the present invention, the process can include after the step of increasing the activity, conducting (or transporting) the remaining portion of the dilute aqueous solution, such as a fermentation broth, to a fermentation vessel. In this embodiment, the remaining portion of the dilute aqueous solution can comprise an impurity and the process further includes removing at least a portion of the impurity from at least a portion of the remaining portion before conducting the remaining portion to the fermentation vessel. Such impurities can be, for example, ethanol, acetate, aldehydes such as butyraldehyde, and short chain fatty acids. In some embodiments, the dilute aqueous solution can include an impurity and the ratio of the impurity to the C3-C6 alcohol in the C3-C6 alcohol-rich liquid phase is greater than the ratio in the water-rich phase. In some embodiments, the ratio of the impurity to the C3-C6 alcohol in the C3-C6 water-rich liquid phase is greater than the ratio in the alcohol-rich phase.

In further embodiments of the invention, the C3-C6 alcohol-rich phase is further processed to increase the value or utility of the phase. Other embodiments of further processing are disclosed in U.S. Patent Application Pub. No. 20090299109, which is incorporated by reference in its entirety. For example, the C3-C6 alcohol-rich phase can be further processed by (i) distilling substantially pure C3-C6 alcohol from the C3-C6 alcohol-rich phase, (ii) distilling an azeotrope of the C3-C6 alcohol from the C3-C6 alcohol-rich phase, (iii) contacting the C3-C6 alcohol-rich phase with a C3-C6 alcohol-selective adsorbent; or (v) combining the C3-C6 alcohol-rich phase with a hydrocarbon liquid that is immiscible in water. In the case of distilling substantially pure C3-C6 alcohol from the C3-C6 alcohol-rich phase, the substantially pure C3-C6 alcohol can have a low proportion of impurities (such as reflected by having a low ratio of impurities to C3-C6 alcohol). For example, the ratio of impurities to C3-C6 alcohol, in the substantially pure C3-C6 alcohol can be less than about 5/95, less than about 2/98, or less than about 1/99. Alternatively the substantially pure C3-C6 alcohol can have a water content of less than about 5 wt %, less than about 1 wt % or less than about 0.5 wt %.

In the case of combining the C3-C6 alcohol-rich phase with a hydrocarbon liquid that is immiscible in water, the resulting combination can form a single uniform phase. Alternatively, in the case of combining the C3-C6 alcohol-rich phase with a hydrocarbon liquid that is immiscible in water, the combination can form a light phase and a heavy phase and the ratio of C3-C6 alcohol to water in the light phase is greater than in the heavy phase. According to an embodiment of the method, the hydrocarbon liquid is a fuel, such as gasoline. According to a related embodiment, a C3-C6 alcohol-enriched fuel is formed by combining a fuel with a C3-C6 alcohol-rich phase, further comprising water. As a result of combining the C3-C6 alcohol selectively transfers into the fuel phase to form said enriched fuel, whereas the majority of the water contained initially in the alcohol-rich phase separates as a water-rich heavy phase, which is separated from the fuel.

An alternative embodiment of these methods to produce a C3-C6 alcohol that includes culturing a microorganism in a fermentation medium to produce the C3-C6 alcohol. The step of culturing is described in detail above. The method further includes increasing the activity of the C3-C6 alcohol in a portion of the fermentation medium and distilling the portion of the fermentation medium to produce a vapor phase comprising water and C3-C6 alcohol and a liquid phase. The steps of increasing the activity and distilling are discussed above in regard to other embodiments of the present invention. The method further includes conducting the liquid phase resulting from the distillation step (the depleted liquid phase) to the fermentation medium. In a preferred embodiment, the portion of the fermentation medium in which the activity of the C3-C6 alcohol is increased comprises microorganisms that remain in the depleted liquid phase and are returned to the fermentation medium for further production of C3-C6 alcohol by the microorganism. In some embodiments, the liquid phase comprises an impurity and the method further includes removing at least a portion of the impurity from at least a portion of the liquid phase before the step of conducting the liquid phase to the fermentation medium. In embodiments of this method, the ratio of the C3-C6 alcohol to water in the portion of the fermentation medium is less than about 10/90 (w/w), less than about 7.5/92.5 (w/w), less than about 5.0/95 (w/w), less than about 2.5/97.5 (w/w), less than about 2/98 (w/w), less than about 1.5/98.5 (w/w), less than about 1/99 (w/w), or less than about 0.5/99.5 (w/w).

The step of distilling may be adiabatic or isothermal. In adiabatic distilling no significant heat transfer takes place between the distillation system and the surroundings, and the pressure of the system is held constant. In isothermal distilling heat transfer is allowed between the distillation system and the surroundings, and the temperature of the system is held constant.

In various embodiments of this method, the enrichment of alcohol from the dilute aqueous solution to the vapor is at least about 5 fold, about 6 fold, about 7 fold, about 8 fold, about 9 fold, about 10 fold, about 11 fold, about 12 fold, about 13 fold, about 14 fold or about 15 fold. The term “enrichment” refers to the ratio of alcohol/water in the condensed vapor divided by the ratio of alcohol/water in the aqueous dilute solution.

Another embodiment of the invention is a method for extraction of a C3-C6 alcohol from an aqueous solution that includes contacting an aqueous solution with an acidic, amine-based extractant. The acidic amine-based extractant can be formed by acidifying an organic amine solution as described above. Upon contact of the aqueous solution with the extractant, the extraction is carried out by mixing the acidic, amine-based extractant with the aqueous solution. The C3-C6 alcohol can be recovered from an extractant phase that forms after contact.

Various aspects of the invention are described in detail in the examples provided below. However, these examples are provided for the purpose of illustration and are not intended to limit the scope of the present invention. Each publication and reference cited herein is incorporated herein by reference in its entirety. While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

### EXAMPLES

### Example 1

This example illustrates the scale-up of an isobutanol production process in accordance with the present invention from lab scale to 1 MM GPY (gallons per year) demonstration scale. An E. coli metabolically engineered in accordance with the teachings of WO 2008/098227 (Gevo2525) to produce isobutanol was propagated through a three fermentor seed train to inoculate a 10,000 L production fermentor. The isobutanol was removed from the culture by vacuum vaporization and recovered by direct contact condensation and liquid-liquid separation.

Gevo2525 was propagated through a three stage seed train, each stage was controlled at 30 C and pH=7. In the first stage, three 3 L shake flasks, the cultures grew to an average optical density (OD₆₀₀ₙₘ) of 6.5. In the second stage, one 50 L fermentor, the culture grew to an OD₆₀₀ₙₘ=7.1. In the final stage, one 500 L fermentor, the OD₆₀₀ₙₘ reached 28 (about 8.1 g cell dry weight per liter). The entire volume of the 500 L fermentor was used to inoculate the 10,000 L production fermentor. For Gevo2525, 1 OD₆₀₀ₙₘ corresponds to approximately 0.45 g cell dry weight per liter.

The culture in the production fermentor was initially grown in aerobic conditions. One hour after inoculation, at an OD₆₀₀ₙₘ=2, IPTG was added to a concentration of 0:1 mM to chemically induce production of enzymes engineered into the microorganism. Approximately 8 hours later, at a cell concentration of OD₆₀₀ₙₘ=12 (cell density of about 5.4 g cell dry weight per liter) and an isobutanol concentration of 6.2 g/L, the fermentor was sparged with Argon to ensure anaerobic conditions. The gas sparge also stripped volatile compounds, including the alcohol product, from the fermentation broth. Alcohol product in the off-gas can be recovered by condensing it from the off-gas.

To maintain the fermentor isobutanol concentration below an inhibitory level during the production phase, the fermentation broth or medium was heated and sent through a scalper for removing at least some gases from the fermentation broth and into a flash tank to recover at least a portion of the alcohol product before being returned to the fermentor. The inlet stream to the scalper was heated from 30 C to 36 C and the scalper was operated at 4 psia while the flash tank was operated at 0.5 psia. The 0.5 psia pressure was generated by two steam eductors arranged in series. The scalper removed most of the dissolved CO₂ from the fermentor broth and decreased the non-condensable load in the flash tank. Aspen Plus® 2006.5 (Aspen Technology, Inc., Burlington, Mass.) modeling estimates that 75% of the CO₂ entering the scalper was removed at 36 C and 4 psia. The residence time in the flash tank was sufficient to reach equilibrium and remove 14% of the broth isobutanol per pass. At 0.5 psia the vapor will be at 11 wt % isobutanol compared with 0.5 wt % in the broth. If inhibitory levels of volatile compounds occurred during the growth phase, the fermentation broth could be recirculated through the flash tank during that stage of the process to remove them.

After the flash tank, the remaining fermentation medium was recirculated to the production fermentor. The recirculation loop (fermentor-preheat-scalper-flash tank-fermentor) ran at 50 gpm.

The flash tank was part of flash tank/direct contact condenser system as illustrated in FIG. 4 and described in the specification. The vapors produced in the flash tank portion of the system were conveyed to the direct contact condenser portion of the system and exposed to a fine spray of recirculated condensate that contains the alcohol product to increase the condensation rate. The recirculated condensate that was used for condensing the vapors was first cooled by a heat exchanger. The remainder of the condensate that was not used as the fine spray was sent to a liquid-liquid separator.

After production in the production fermentor was complete, the spent broth was sent to a beer still. iBuOH in the spent broth was recovered in the beer still and the production microorganisms were inactivated.

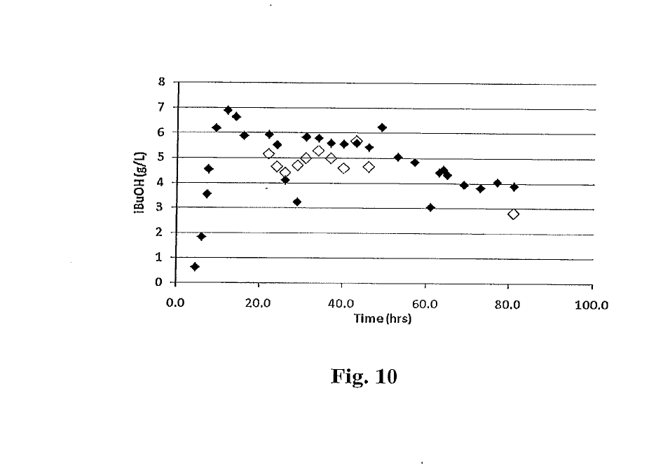

With reference to FIG. 10, the isobutanol concentration in the fermentor broth and in the post flash broth is illustrated. It can be seen that the flash tank removed approximately 15%-20% of the broth iBuOH before the broth was returned to the fermentor.

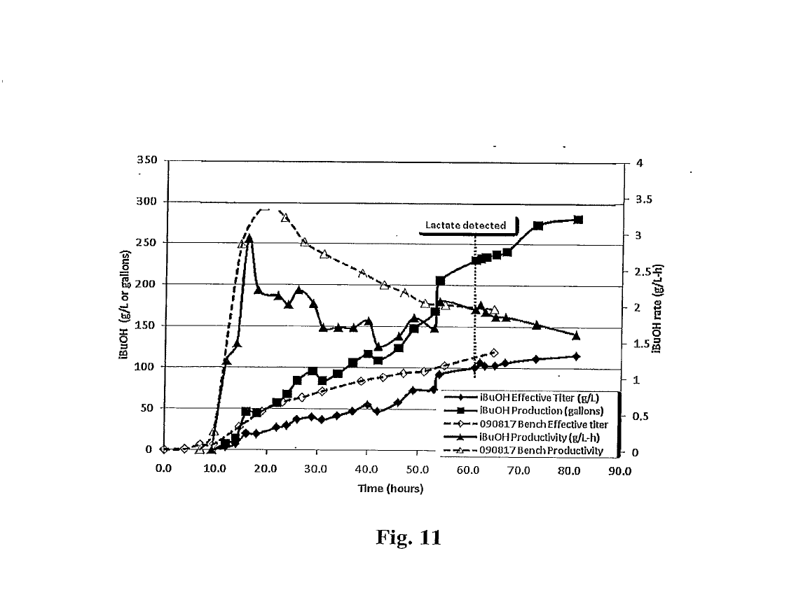

Isobutanol production was calculated for the anaerobic phase based on glucose consumption assuming 90% of the 0.41 g isobutanol per g glucose theoretical yield and accounted for the glucose consumed by a contaminating microbe to produce lactate, the major byproduct. FIG. 11 shows that the effective titer and productivity were comparable to a previous bench scale experiment. The results of this fermentation run and recovery are shown below in Table 1.

TABLE 1
Summary of Isobutanol Production

| | |
|---|---|
| Effective Titer of Isobutanol* | 115 |
| (g/L) | |
| Total Gallons | 280 |
| (Gallons) | |
| Volumetric Productivity of Isobutanol | 1.9 |
| (g/L-h) | |
| Production Time | 70 |
| (hours) | |
| Initial Productivity of Isobutanol | 2.9 |
| (g/L-hr) | |
| Run Time for Initial Productivity | 6 |
| (h) | |
| Overall Productivity of Isobutanol | 1.6 |
| (g/L-hr) | |

*Total grams of isobutanol produced per liter of fermentation broth

### Example 2

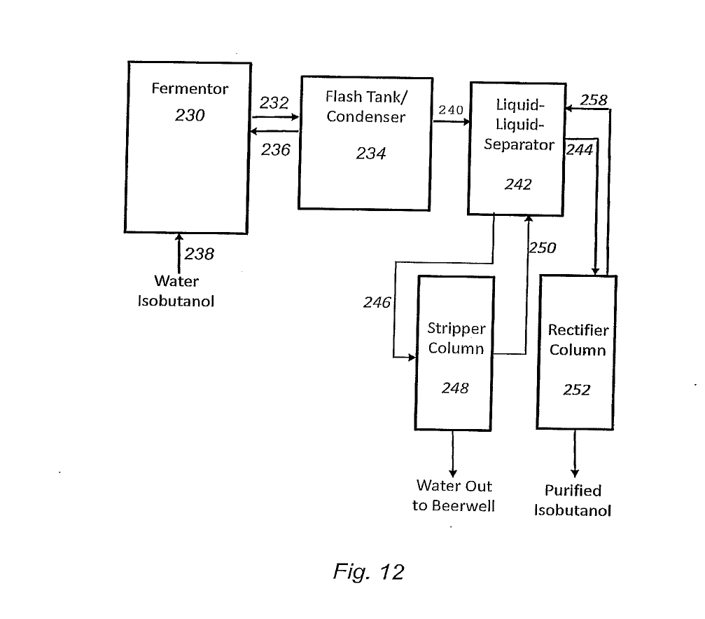

This example illustrates the removal, recovery and purification of isobutanol from solution to simulate operation of a high productivity fermentation (2.8 g/L-hr) in accordance with the present invention. From a 2 wt % isobutanol solution, a removal rate of 37.4 kg/hr was achieved. Purification of the recovered isobutanol by distillation using a two column system resulted in a moisture content in the butanol product of less than 1%. The process flow of this example is shown in FIG. 12.

A 45,000 L working volume fermentor 230 was filled with 13,400 L of water. Isobutanol was added via 238 to a final concentration of 2 wt %. The solution was heated and sent through a scalper for removing at least some gases in the fermentation broth and into a flash tank portion of a flash tank/direct contact condenser system 234 via 232 to recover at least a portion of the alcohol product before being returned to the fermentor via 236. The inlet stream to the scalper (not shown) was heated from 30 C to 36 C and the scalper was operated at 4 psia while the flash tank was operated at 0.5 psia. The 0.5 psia pressure was generated by two steam eductors in series. The scalper removed most of the dissolved CO₂ from the fermentor broth and decreased the non-condensable load in the flash tank. Aspen Plus® 2006.5 modeling estimates that 75% of the CO₂ entering the scalper was removed at 36 C and 4 psi a. The residence time in the flash tank was sufficient to reach equilibrium and remove 15% of the isobutanol per pass. At 0.5 psia, the vapor was at 41 wt % (based on modeling the system) compared with 2 wt % in the solution. The recirculation loop through the flash tank was run at 55 gpm and achieved a fermentor turnover Tate of 1.1 volumes/hour. Additional isobutanol was fed to the fermentor at 34 kg/hr to simulate isobutanol production by an active fermentation.

The 41 wt % isobutanol vapor was condensed by direct contact with sprayed liquid on the condensate side of the flash tank flash tank/direct contact condenser system 234. The condensate was fed to the liquid-liquid separator 242 via 240 where the isobutanol-rich light phase and the water-rich heavy phases separated. The heavy phase was fed to the stripper column 248 via 246 which was operated at 10 psia with condensed overhead vapors containing isobutanol being sent to the liquid-liquid separator 242 via 250. The light phase product from the liquid-liquid separator was sent to the rectifier column 252 via 254 which was operated at 4-5 psia. The overhead vapors from the rectifier column containing water and alcohol were sent to the liquid-liquid separator 242 via 258. The purified isobutanol produced at the bottom of the rectifier column was collected via 256 and analyzed. Results of this simulation run are shown below in Table 2.

TABLE 2
Simulation Run Summary Performance

| | | Steady State | | | Average Bottom | |
|---|---|---|---|---|---|---|
| | Operating | Average | | Aspen | [iBuOH] | |
| | P | Top | Bottom | Bottom | | Mass |
| Column | [PSIA] | [° F.] | [° F.] | [° F.] | g/L | Fraction |
| Stripper | 10 | 191 | 194 | 193 | 0.93 | 0.0009 |
| Rectifier | 4.9 | 150 | 174 | 175 | 812.9 | 0.99 |

### Example 3

This example illustrates the production benefit of increased aeration in a fermentation broth during the production phase when combined with vacuum removal in accordance with the present invention. A 2-L DasGip fermentor was used with a 400 ml flash vessel. The fermentor was operated with a yeast production microorganism at 30 C, pH=6.0 with an initial volume of 1.1 L. The flash vessel was operated at 36 C at a vacuum level of 0.7-0.9 psia, the fermentation broth was recirculated to the flash vessel when the broth isobutanol titer was approximately 3 g/L. The fermentation media was replaced with fresh media when acetate levels increased, approximately every 24-48 hours.

The fermentor was run under aerobic conditions for the first 14 hours after inoculation with oxygen transfer rate (“OTR”) reaching 15-16 mM/L-h to increase the density of the microorganism and with little production of alcohol product. To increase production, aeration was reduced with a target OTR of 5 mM/L-h and a volumetric productivity of 0.24 g/L-h was achieved. Overall volumetric productivity steadily decreased from the maximum rate of 0.24 g/L-h at 217 hours to 0.21 g/L-h at 349 hours. Aeration was then increased to an OTR of approximately 8 mM/l-h for the duration of the fermentation and the productivity again reached 0.24 g/l-h.

This example illustrates that productivity can be increased by increasing the OTR during a fermentation during a production phase.

### Example 4

This example illustrates the removal and recovery of isobutanol from fermentation broth using an adiabatic flash. Aspen Plus® 2006.5 was used to generate equilibrium data for a fermentation broth pumped to and from a flash vessel and flashed at 35.0 and 37.0 C at varying flash pressures. The Non-Random Two Liquid (NRTL) thermodynamic model within Aspen Plus® was utilized. The system is shown schematically in FIG. 14.

The stream from the fermentor was fixed at an operating pressure of 1 atm absolute and a composition (mass fraction) of 0.9789 water, 0.0011 carbon dioxide, and 0.0200 isobutanol was assumed to flow on a 1000 kmol/hr basis into a scalper operating adiabatically at 4 psia. The results for these conditions, shown in the Table 3 below, indicate that high percentages of isobutanol are removed from the broth as indicated by the percent of isobutanol removed per pass through the flash system and that a vapor enrichment occurs as indicated by the concentration factor using an adiabatic flash system.

TABLE 3

| | | | | | Broth Isobutanol | | | | | |
|---|---|---|---|---|---|---|---|---|---|---|
| | | | | | Concentration | | | Isobutanol | Flash Condensate | |
| | | | | | Broth | Broth | | Removed | Flash | |
| Scalp/Flash Conditions | | | | | into | into | Broth | From Broth | Cond. | |
| T_{IN SCALP} | T_{IN FLASH} | T_{OUT FLASH} | P_{SCALP} | P_{FLASH} | Scalper | Flash | Return | Per Pass | Conc. | Conc. |
| C | C | C | bar | bar | g/L | g/L | g/L | % | g/L | Factor |
| 35.0 | 34.8 | 24.6 | 0.276 | 0.034 | 19.61 | 19.57 | 13.50 | 31.0 | 318.5 | 23.6 |
| 35.0 | 34.8 | 31.0 | 0.276 | 0.052 | 19.61 | 19.57 | 16.80 | 14.2 | 294.6 | 17.5 |
| 37.0 | 36.8 | 24.7 | 0.276 | 0.034 | 19.57 | 19.53 | 12.60 | 35.5 | 305.4 | 24.2 |
| 37.0 | 36.8 | 31.2 | 0.276 | 0.052 | 19.57 | 19.53 | 15.74 | 19.4 | 347.8 | 22.1 |

This example demonstrates that adiabatic flash is an effective method for removing isobutanol from a fermentation broth.

### Example 5

This example illustrates the removal and recovery of isobutanol from fermentation broth using an isothermal flash. Aspen Plus® 2006.5 was used to generate equilibrium data for a fermentation broth pumped to and from a flash vessel and flashed at 35.0 and 37.0 C at varying flash pressures. The Non-Random Two Liquid (NRTL) thermodynamic model within Aspen Plus® was utilized.

The stream from the fermentor was fixed at an operating pressure of 1 atm absolute and a composition (mass fraction) of 0.9789 water, 0.0011 carbon dioxide, and 0.0200 isobutanol was assumed to flow on a 1000 kmol/hr basis into a scalper operating adiabatically at 4 psia. The results for these conditions, shown in Table 4 below, indicate that high percentages of isobutanol are removed from the broth as indicated by the percent of isobutanol removed per pass through the flash system and that a vapor enrichment occurs as indicated by the concentration factor using an isothermal flash system.

TABLE 4

| | | | | | Broth Isobutanol | | | | | |
|---|---|---|---|---|---|---|---|---|---|---|
| | | | | | Concentration | | | Isobutanol | Flash Condensate | |
| | | | | | Broth | Broth | | Removed | Flash | |
| Scalp/Flash Conditions | | | | | into | into | Broth | From Broth | Cond. | |
| T_{IN SCALP} | T_{IN FLASH} | T_{OUT FLASH} | P_{SCALP} | P_{FLASH} | Scalper | Flash | Return | Per Pass | Conc. | Conc. |
| C | C | C | bar | bar | g/L | g/L | g/L | % | g/L | Factor |
| 35.0 | 34.8 | 35.0 | 0.276 | 0.066 | 19.61 | 19.57 | 17.60 | 10.1 | 369.9 | 21.0 |
| 35.0 | 34.8 | 35.0 | 0.276 | 0.062 | 19.61 | 19.57 | 12.21 | 37.6 | 294.6 | 24.1 |
| 37.0 | 36.8 | 37.0 | 0.276 | 0.069 | 19.57 | 19.53 | 11.72 | 40.0 | 285.4 | 24.4 |
| 37.0 | 36.8 | 37.0 | 0.276 | 0.066 | 19.57 | 19.53 | 4.99 | 74.5 | 149.8 | 30.1 |

This example demonstrates that isothermal flash is an effective method for removing isobutanol from a fermentation broth.

### Example 6

This example illustrates the removal and recovery of isobutanol from fermentation broth using a four stage column utilizing an isothermal flash on the fourth stage. Aspen Plus® 2006.5 was used to generate equilibrium data for a fermentation broth pumped to and from a flash vessel and flashed at 35.0 and 37.0 C at the indicated column pressures. The Non-Random Two Liquid (NRTL) thermodynamic model within Aspen Plus® was utilized.

The stream from the fermentor was fixed at an operating pressure of 1 atm absolute and a composition (mass fraction) of 0.9789 water, 0.0011 carbon dioxide, and 0.0200 isobutanol was assumed to flow on a 1000 kmol/hr basis into a scalper operating adiabatically at 4 psia. The results for these conditions, shown in Table 5 below, indicate that high percentages of isobutanol are removed from the broth as indicated by the percent of isobutanol removed per pass through the lower, fourth stage of the column and that a vapor enrichment occurs as indicated by the concentration factor using this configuration.

TABLE 5

| | | | | | Broth Isobutanol | | | | | |
|---|---|---|---|---|---|---|---|---|---|---|
| | | | | | Concentration | | | Isobutanol | Flash Condensate | |
| | | | | | Broth | Broth | | Removed | Flash | |
| Scalp/Flash Conditions | | | | | into | into | Broth | From Broth | Cond. | |
| T_{IN SCALP} | T_{IN FLASH} | T_{OUT FLASH} | P_{SCALP} | P_{FLASH} | Scalper | Flash | Return | Per Pass | Conc. | Conc. |
| C | C | C | bar | bar | g/L | g/L | g/L | % | g/L | Factor |
| 35.0 | 34.8 | 34.8 | 0.276 | 0.058 | 19.61 | 19.57 | 5.39 | 69.9 | 467.6 | 79.4 |
| 37.0 | 36.8 | 36.8 | 0.276 | 0.065 | 19.57 | 19.53 | 5.93 | 69.6 | 463.9 | 78.2 |

This example demonstrates that a multistage isothermal flash is an effective method for removing isobutanol from a fermentation broth.

### Example 7

This example illustrates the fermenter turnover rate required to maintain the isobutanol titer in a fermenter at equilibrium for adiabatic and isothermal flash conditions at varying isobutanol productivities. By multiplying the fermenter turnover rate by a given fermenter volume, the recycle pumping rate required to maintain a constant fermenter titer is obtained. The titers for the broth into flash and broth return were generated as explained in previous Examples 4 and 5 (last lines of Tables 3 and 4) for adiabatic and isothermal flash conditions.

The results shown in Table 6 below indicate that a lower fermenter turnover rate and thus a lower recycle pumping rate are required for an isothermal flash versus an adiabatic flash at a given productivity.

TABLE 6

| | | Broth | | Fermenter |
|---|---|---|---|---|
| | | into | Broth | Turnover |
| Flash | Productivity | Flash | Return | Rate |
| Condition | g/L Hr | g/L | g/L | l/Hr |
| Adiabatic | 0.5 | 19.53 | 15.74 | 0.132 |
| Flash | 2.0 | 19.53 | 15.74 | 0.528 |
| | 3.0 | 19.53 | 15.74 | 0.792 |
| | 5.0 | 19.53 | 15.74 | 1.319 |
| Isothermal | 0.5 | 19.53 | 4.99 | 0.034 |
| Flash | 2.0 | 19.53 | 4.99 | 0.138 |
| | 3.0 | 19.53 | 4.99 | 0.206 |
| | 5.0 | 19.53 | 4.99 | 0.344 |

This example demonstrates that an isothermal flash requires a lower fermenter turnover rate when compared to an adiabatic flash.

The principles, preferred embodiments and modes of operation of the present invention have been described in the foregoing specification. The invention which is intended to be protected herein should not, however, be construed as limited to the particular forms disclosed, as these are to be regarded as illustrative rather than restrictive. Variations and changes may be made by those skilled in the art without departing from the spirit of the present invention. Accordingly, the foregoing best mode of carrying out the invention should be considered exemplary in nature and not as limiting to the scope and spirit of the invention as set forth in the appended claims.

1-7. (canceled)

8. The method of claim 24, wherein the gases comprise carbon dioxide.

9. (canceled)

10. The method of claim 8, wherein at least about 75% of the carbon dioxide is removed during the step of removing.

11. The method of claim 8, wherein at least about 85% of the carbon dioxide is removed during the step of removing.

12. The method of claim 8, wherein at least about 90% of the carbon dioxide is removed during the step of removing.

13. (canceled)

14. The method of claim 8, wherein the step of removing comprises reducing pressure to a pressure of between about 1 psia and about 10 psia.

15. (canceled)

16. The method of claim 8, wherein the removed carbon dioxide is conducted to a fermentation unit for pH control, vented, or combinations thereof.

17. The method of claim 8, further comprising treating the gases to remove the C3-C6 alcohol and venting the gases.

18-23. (canceled)

24. A method to produce a C3-C6 alcohol from a fermentation medium comprising microorganisms, gases and the C3-C6 alcohol, comprising:
a. culturing a microorganism in a fermentation medium to produce the C3-C6 alcohol;
b. removing at least a portion of the gases from the fermentation medium;
c. distilling the portion of the fermentation medium to form a vapor phase comprising water and the C3-C6 alcohol and a liquid phase;
d. condensing the vapor phase by contacting it with a solution comprising the C3-C6 alcohol, and
e. conducting the liquid phase to the fermentation medium.

25. (canceled)

26. The method of claim 24, wherein the solution comprising the C3-C6 alcohol is sprayed into the vapor phase formed in step (c).

27. The method of claim 24, wherein the solution comprising the C3-C6 alcohol comprises the condensate of step (d).

28. The method of claim 27, wherein the condensate is cooled prior to being contacted with the vapor phase formed in step (c).

29. The method of claim 24, wherein the step of forming the vapor phase and the step of condensing the vapor phase are conducted in a single vessel.

30. The method of claim 29, wherein the vessel comprises a weir defining first and second fluid containing portions, wherein the first fluid containing portion is adapted to receive the fermentation medium, and the second fluid containing portion is adapted to receive the condensed vapor phase.

31. The method of claim 30, wherein the first fluid containing portion comprises a conduit for conducting the fermentation medium into the first fluid containing portion and a conduit for conducting the fermentation medium out of the first fluid containing portion, wherein the content of the C3-C6 alcohol in the fermentation medium that is conducted out of the first fluid containing portion is less than that of the fermentation medium that is conducted into the first fluid containing portion.

32. The method of claim 30, wherein the second fluid containing portion comprises a conduit for conducting the condensed vapor phase out of the second fluid containing portion.

33. A flash tank/direct contact condenser system for increasing the concentration of a C3-C6 alcohol in an aqueous solution comprising:
a. a vessel;
b. means for introducing a stream of aqueous solution comprising the C3-C6 alcohol into the vessel;
c. means for subjecting the stream of aqueous solution comprising the C3-C6 alcohol to reduced pressure to form a vapor comprising the C3-C6 alcohol;
d. means for contacting the vapor comprising the C3-C6 alcohol with a solution comprising the C3-C6 alcohol to form a condensate comprising condensed vapor of the C3-C6 alcohol, wherein the concentration of the C3-C6 alcohol in the condensate is greater than the concentration of the C3-C6 alcohol in the first stream of aqueous solution.

34. The flash tank/direct contact condenser system of claim 33, wherein the vessel comprises two fluid containing compartments or portions that are separated by a weir, wherein the weir divides the compartments or portions at the bottom of the vessel.

35. The flash tank/direct contact condenser system of claim 34, wherein the means (c) comprises a means for creating a vacuum.

36. The flash tank/direct contact condenser system of claim 34, wherein the means (d) comprises a spray nozzle.

37-54. (canceled)

55. A method to produce a C3-C6 alcohol, comprising:
a. culturing a microorganism in a fermentation medium to produce the C3-C6 alcohol;
b. introducing a gas comprising oxygen into the fermentation medium during step (a) at an oxygen transfer rate (OTR) of less than about 20 mmoles of oxygen per liter of fermentation medium per hour;
c. distilling the portion of the fermentation medium to produce a vapor phase comprising water and C3-C6 alcohol and a liquid phase, and
d. conducting the liquid phase to the fermentation medium.

56-78. (canceled)
